(19) United States
(12) Patent Application Publication
(10) Pub. No.: US 2026/0337593 A1
(43) Pub. Date: Oct. 29, 2026

(54) METHOD AND MEANS FOR ENGINEERING NON-RIBOSOMAL PEPTIDES

(71) Applicant: JOHANN WOLFGANG GOETHE-UNIVERSITÄT FRANKFURT AM MAIN, Frankfurt am Main (DE)

(72) Inventors: Helge B. BODE, Marburg (DE); Kenan BOZHÜYÜK, Marburg (DE); Carsten KEGLER, Oberursel (DE); Alexander RILL, Kelkheim (DE)

(73) Assignee: JOHANN WOLFGANG GOETHE-UNIVERSITÄT FRANKFURT AM MAIN, Frankfurt am Main (DE)

(21) Appl. No.: 19/103,757

(22) PCT Filed: Aug. 16, 2023
(86) PCT No.: PCT/EP2023/072624

§ 371 Date: Feb. 13, 2025

(30) Foreign Application Priority Data
Aug. 16, 2022 (EP) 22190641.5

(51) Int. Cl. C12N 9/00; C12N 9/10

(52) U.S. Cl. CPC C12N 9/93; C12N 9/1029; C07K 2319/00

(57) ABSTRACT

The invention pertains to a strategy for combining elements of non-ribosomal peptide synthases (NRPS) as well as polyketide synthases (PKS) into functioning enzymes for facilitating the generation of peptides and polyketides of any sequence. The invention is based upon the use of a conserved sequence motif within the T-domain (or T-domain like) gene of NRPS/PKS which is used according to the invention as fusion point for the generation of new (and artificial) NRPS/PKS enzymes that may generate any peptide or polyketide of interest. The invention provides the artificial NRPS/PKS of the invention, methods and means for their generation, as well as nucleic acid libraries encoding various modules that can be used to design NRPS/PKS for any peptide sequence of interest.

### FIELD OF THE INVENTION

The invention pertains to a strategy for combining elements of non-ribosomal peptide synthases (NRPS) as well as polyketide synthases (PKS) into functioning enzymes for facilitating the generation of peptides and polyketides of any sequence. The invention is based upon the use of a conserved sequence motif within the T-domain (or T-domain like) gene of NRPS/PKS which is used according to the invention as fusion point for the generation of new (and artificial) NRPS/PKS enzymes that may generate any peptide or polyketide of interest. The invention provides the artificial NRPS/PKS of the invention, methods and means for their generation, as well as nucleic acid libraries encoding various modules that can be used to design NRPS/PKS for any peptide sequence of interest.

### DESCRIPTION

The invention relates to a system for the generation and expression of chimeric nonribosomal peptide synthetases (NRPSs), polyketide synthases (PKSs), or NRPS/PKS hybrid synth(et)ases. NRPSs, PKSs or their hybrids are large multifunctional modular multidomain proteins or complexes, producing a wide variety of clinically relevant scaffolds. Their expression and modification for peptide production and product alteration, respectively, present many difficulties. The present invention relates to a novel evolutionary inspired strategy (EIS) for functionally recombining, expressing, and producing artificial nonribosomal peptides (NRPs), polyketides (PKs), and hybrids thereof. The EIS method leverages in silico detected and in vivo verified splicing positions enabling both, the rational modification of targeted biosynthetic gene clusters (BGCs) and the de novo assembly of novel artificial/synthetic BGCs. The invention discloses specifically defined splicing positions, protein fragments, and recombination strategies of such a molecular synthetic biology assembly kit, as well as their coding nucleic acids. Also disclosed are the cloning strategy of the nucleotide fragments of the invention and their use in preparing functional NRPS/PKS enzymes to generate a library of novel bioactive NRP/PK scaffolds.

Reprogramming biosynthetic assembly lines (NRPS, PKS, NRPS/PKS hybrids) is of intense interest. This is unsurprising as the scaffolds of many antibiotics (penicillins4) immune suppressants (rapamycin5), and anti-cancer (bleomycin6) therapeutics in clinical use are produced by such pathways.

Essentially, NRPS7 and PKS8 modular megasynth(et)ases give rise to highly functionalised biopolymers from a wide variety of monomers, referred to as extender units. Hundreds of extender units have been reported9, typically derived from amino acids in the case of NRPS10, 11 or malonate in the case of PKSs12. They are likened to assembly-line processes due to their hierarchical and modular structures. Multiple repeating modules of enzymatic domains catalyse the incorporation of an extender unit into the growing chain, along with any programmed additional chemical modifications, before transferring the elongated chain to the next module. The archetypical minimal assembly-line module consists of three ‘core’ domains. Firstly, a domain for selecting and activating an extender unit, the adenylation (A) domains for NRPSs or the acyltransferase (AT) domains for PKSs. The activated substrate is then covalently attached to a prosthetic phosphopantetheine group of a small peptidyl carrier protein (PCP; NRPSs), also denoted as thiolation (T) domain, or acyl carrier protein (ACP; PKSs) domain. Finally, the condensation (C; NRPSs) domains or the ketosynthase (KS; PKSs) then link the covalently bound substrates to the growing peptide or polyketide chain. Although their modular nature provides a direct relationship between the sequence of enzymatic domains and the product chemical structure13, 14, efforts to reprogram them rationally have had limited success in the past15.

Since 1995, when Marahiel et al. (WO200052152) were able to show that it is possible to recombine NRPS through exchanging adenylation-thiolation didomains, NRPS research came into focus (Marahiel et al. 1995). During the last two decades, there have been a lot of attempts to reprogram NRPS. Based on the crystal structure of the phenylalanine activating domain PheA (PDB-ID: 1AMU) Stachelhaus et al. were able to elucidate the specificity conferring AAs in the catalytic center (Conti et al. 1997, Stachelhaus et al. 1999). With this specificity conferring code, denoted as Stachelhaus-code it is possible to predict and to change substrate specificities of A domains in vitro, (Khurana et al. 2010, Rausch et al. 2005, Röttig et al. 2011, Kries et al. 2014). The most obvious disadvantage of this attempt is its inapplicability in vivo. One major reason for this drawback is that C and TE domains also have selectivities resulting in substrate incompatibilities (Belshaw et al. 1999; Trauger et al. 2000; Tseng et al. 2002).

A further attempt (WO200130985, Marahiel et al.) to vary known NRPS biosynthetic clusters is based on the exchange of single domains, didomains or whole modules and the knowledge of exactly defined borders (linkers) between individual domains. With this invention it was only possible to alter a few NRPSs successfully by introduction of additional modules or deleting them. However, it never was possible to produce totally artificial NRPSs from the artificial de novo combination of modules or domains. This would result in new NRPS not present in nature that would also produce new peptides. The problem of such exchanges or combinations always was the uncertainty concerning the compatibility of modules and/or domains between each other. The shortcomings resulting from the lack of a solution to the problem mentioned above is illustrated by the fact that only very few peptide derivatives have been designed by this approach.

Another attempt (WO2007014076, Walsh et al.) to vary known NRPS biosynthetic clusters is based on mutagenesis of so called “assembly lines” other word for synthases. Mutagenesis of genes of NRPS is not subject of the present invention although the present inventive methods can be combined with a mutagenesis that will alter the generated NRPS and cause altered peptide synthesis. This mutagenesis could be useful for increasing the diversification of NRPS libraries and the NRPS clone numbers in the library.

Thus, it is an object of the invention to provide alternative approaches for facilitating the design and expression of artificial NRPS/PKS for peptide and polyketide synthesis.

### BRIEF DESCRIPTION OF THE INVENTION

Generally, and by way of brief description, the main aspects of the present invention can be described as follows:

In a first aspect, the invention pertains to a chimeric protein for the production of nonribosomal peptides, comprising at least one chimeric thiolation domain (T-domain (PKS acyl carrier protein (ACP) or NRPS petidyl carrier (PCP))) consisting of a first T-domain (PKS acyl carrier protein (ACP) or NRPS petidyl carrier (PCP)) segment directly adjacent to a second T-domain (PKS acyl carrier protein (ACP) or NRPS petidyl carrier (PCP)) segment, wherein the first T-domain (PKS acyl carrier protein (ACP) or NRPS petidyl carrier (PCP)) segment comprises an amino acid sequence from an N-terminal part of a T-domain (PKS acyl carrier protein (ACP) or NRPS petidyl carrier (PCP)) amino acid sequence, and wherein the second T-domain (PKS acyl carrier protein (ACP) or NRPS petidyl carrier (PCP)) segment comprises an amino acid sequence from a C-terminal part of a T-domain (PKS acyl carrier protein (ACP) or NRPS petidyl carrier (PCP)) amino sequence; wherein the chimeric thiolation domain is a fully functional T-domain (PKS acyl carrier protein (ACP) or NRPS petidyl carrier (PCP)); characterized in that the amino acid sequences of the parts of the T-domain (PKS acyl carrier protein (ACP) or NRPS petidyl carrier (PCP)) amino acid sequences of the first and the second T-domain (PKS acyl carrier protein (ACP) or NRPS petidyl carrier (PCP)) segments are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.

In an alternative first aspect, the invention further pertains to a method for producing a chimeric non-ribosomal peptide synthetase (NRPS)- and/or polyketide (PKS)-module, the method comprising:
(a) Providing a first NRPS/PKS module sequence comprising at least a first T-domain (T-domain (PKS acyl carrier protein (ACP) or NRPS petidyl carrier (PCP)) sequence;
(b) Providing a second NRPS/PKS module sequence comprising at least a second T-domain sequence;
(c) Optionally, aligning the first T-domain sequence to the second T-domain sequence to identify at least one T-domain fusion point;
(d) Fusing by cloning directly adjacent to each other into a genetic construct a first partial sequence to a second partial sequence to obtain a chimeric module sequence, wherein
   (i) The first partial sequence is a nucleotide sequence encoding an amino acid sequence of the first NRPS/PKS module sequence located directly N-terminal to the identified T-domain fusion point; and
   (ii) The second partial sequence is a nucleotide sequence encoding an amino acid sequence of the second NRPS/PKS module sequence located directly C-terminal to the identified T-domain fusion point;
   and thereby producing a genetic construct comprising a chimeric NRPS- and/or PKS-module.

In a second aspect, the invention pertains a system for the generation of chimeric nonribosomal peptide synthetases (NRPSs), a chimeric polyketide synthase (PKSs), or a chimeric NRPS/PKS hybrid, comprising at least two system units:
(a) a first system unit comprising at least the first T-domain (PKS acyl carrier protein (ACP) or NRPS petidyl carrier (PCP)) segment recited in any one of the other aspects and embodiments of this aspects, and
(b) a second system unit comprising at least the second T domain segment recited in any one of the other aspects and embodiments of this aspects;
   wherein each system unit may comprise one or more of further NRPS/PKS domains, and wherein the chimeric non-ribosomal peptide synthetases (NRPSs), the chimeric polyketide synthase (PKSs), or the chimeric NRPS/PKS hybrid is obtainable by fusing the first and the second system unit such that the N-terminal T-domain (PKS acyl carrier protein (ACP) or NRPS petidyl carrier (PCP)) amino acid sequence of the first system unit is directly fused to the C-terminal T-domain (PKS acyl carrier protein (ACP) or NRPS petidyl carrier (PCP)) amino acid sequence of the second system unit.

In a third aspect, the invention pertains to a method for the generation of chimeric nonribosomal peptide synthetases (NRPSs), a chimeric polyketide synthase (PKSs), or a chimeric NRPS/PKS hybrid, the method comprising the steps of:
(a) Providing a first NRPS or PKS gene sequence from a first microorganism species,
(b) Providing a second NRPS or PKS gene sequence from a second microorganism species, and
(c) Combining at least one part of the first NRPS or PKS gene sequence with at least one part of the second NRPS or PKS gene sequence to obtain a chimeric gene sequence by fusing both sequences to each other such that at least one fusion point is located within a T domain, and wherein the fusion product comprises a T domain consisting of an N-terminal sequence of a T domain of the first NRPS or PKS gene sequence and a C-terminal sequence of a T domain of the second NRPS or PKS gene sequence, and
(d) Optionally, expressing the chimeric gene sequence to obtain the chimeric nonribosomal peptide synthetases (NRPSs), a chimeric polyketide synthase (PKSs), or a chimeric NRPS/PKS hybrid.

In a fourth aspect, the invention pertains to a method for combining, or producing a combination of, at least two NRPS/PKS modules from two different NRPS/PKS genes or from two separated locations within the same NRPS/PKS gene, the method comprising the steps of:
(a) Identifying a first target NRPS/PKS module that is to be combined directly N-terminally to a second target NRPS/PKS module;
(b) Identifying a second target NRPS/PKS module that is to be combined directly C-terminally to a second target NRPS/PKS module, and wherein the second target NRPS/PKS module is selected from a module that is located within a NRPS/PKS gene C-terminally to a third non-target NRPS/PKS module;
(c) Combining the first target NRPS/PKS module with the second target NRPS/PKS module by directly attaching an N-terminal part of the T domain of the first NRPS/PKS module with a C-terminal part of a T domain of the third non-target NRPS/PKS module;
   Wherein the combination of the N-terminal part of the T domain of the first NRPS/PKS module with the C-terminal part of a T domain of the third non-target NRPS/PKS module constitute a full 30 and functional T-domain (PKS acyl carrier protein (ACP) or NRPS petidyl carrier (PCP)).

In a fifth aspect, the invention pertains to a combination product obtained by the method of the fourth aspect.

In a sixth aspect, the invention pertains to a method for the generation of a nucleic acid construct encoding a system unit recited in the other aspects and embodiments of this aspects, the method comprising a step of amplifying a sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S, wherein x is any amino acid.

In a seventh aspect, the invention pertains to a method for generating a library of nucleic acid constructs each encoding a system unit recited in of the other aspects and embodiments of this aspects, wherein the method comprises a step of amplifying a first sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S; and wherein the method comprises a second amplification of at least a second sequence stretch of an NRPS/PKS gene using the degenerate primer pair from the same or one or more different NRPS/PKS genes.

In an eighth aspect, the invention pertains to a system of genetic constructs for the expression of a chimeric NRPS/PKS, wherein the system comprises at least one genetic construct comprising a chimeric NRPS- and/or PKS-module as produced by a method of the previous aspects.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

### DEFINITIONS

The term “partial domain” or “partial C or C/E domain” or similar expression shall refer to nucleic acid sequence encoding for, or a protein sequence of, an NRPS C or C/E do-main which is incomplete (not full length). The term therefore describes a C or C/E do-main sequence which does not comprise both donor and acceptor sites of an NRPS C or C/E domain.

By “assembly” is meant a set of domains. A plurality of assembly comprises an NRPS. One or more polypeptides may comprise a module. Combinations of modules can catalyze a series of reactions to form larger molecules. In one example, a module may comprise a C (condensation) domain, an A (adenylation) domain, and a peptidyl carrier protein domain.

For more structural information on A domains, C domains, didomains, domain-domain interfaces and complete modules see Conti et al. (1997), Sundlov et al. (2013), Samel et al. (2007), Tanovic et al. (2008), Strieker and Marahiel (2010), Mitchell et al. (2012) and Tan et al. (2015).→Sind das Zitate aus einem anderen Patent? Diese Liste könnte man durchaus updaten, z.b. mit Drake et al., Izore et al., Reimer et al., NRPS Review: Süssmuth & Mainz

By “initiation module” is meant a N-terminal module which is capable of providing a first monomer to another module (e.g., an elongation or termination module). In some in-stances the other module is not the second but any of the C-terminally following modules (as is the case for the Nocardicin NRPS): In the case of an NRPS, an initiation module comprises, for example, an A (adenylation) domain and a PCP (peptidyl carrier protein) or T (thiolation) domain. The initiation module may also contain a starter C domain and/or an E (epimerization) domain. In the case of a PKS, a possible initiation module comprises an AT (acetyltransferase) domain and an acyl carrier protein (ACP) domain. Initiation modules are preferably at the amino terminus of a polypeptide of the first module of an assembly line, and each assembly line preferably contains one initiation module.

By “elongation module” is meant a module which adds a monomer to another monomer or to a polymer. An elongation module may comprise a C (condensation), Cy (heterocy-clization), E, C/E, MT (methyltransferase), A-MT (combined adenylation and methylation domain), Ox (oxidase), or Re (reductase) domain; an A domain; or a T domain. An elongation domain may further comprise additional E, Re, DH (dehydration), MT, NMet (N-methylation), AMT (Aminotransferase), or Cy domains. Additionally, an elongation module might be of PKS origin comprising the respective domains (ketosynthase (KS), acyltransferase (AT), ketoreductase (KR), dehydratase (DH), enoylrductase (ER), acyl carrier protein (ACP) or thiolation (T)) connecting an amino acid building block with a carboxylic acid building block.

By “termination module” is meant a module that releases the molecule (e.g., an NRP, PK, or combination thereof) from the assembly line. The molecule may be released by, for example, hydrolysis or cyclization. Termination modules may comprise a TE (thioesterase), Cterm, or Re domain. The termination module is preferably at the carboxy terminus of a polypeptide of an NRPS or PKS. The termination module may further comprise additional enzymatic activities (e.g., oligomerase activity).

By “domain” is meant a polypeptide sequence, or a fragment of a larger polypeptide sequence, with one or more specific enzymatic activities (i.e. C/E domains have a C and a E function in one domain) or another conserved function (i.e. as tethering function for an ACP or T domain). Thus, a single polypeptide may comprise multiple domains. Multiple domains may form modules. Examples of domains include C (condensation), Cy (heterocyclization), A (adenylation), T (thiolation), TE (thioesterase), E (epimerization), C/E (condensation/epimerization), MT (methyltransferase), Ox (oxidase), Re (reduc-tase), KS (ketosynthase), AT (acyltransferase), KR (ketoreductase), DH (dehydratase), and ER (enoylreductase).

By “non-ribsomally synthesized peptide,” “non-ribosomal peptide,” or “NRP” is meant any polypeptide not produced by a ribosome. NRPs may be linear, cyclized or branched and contain acyl chains?-->lipopeptides?, aldehydes?, amines?, proteinogenic, natural or non-natural amino acids, or any combination thereof. NRPs include peptides produced in an assembly line like manner (=modular character of the enzyme system allowing a stepwise addition of building blocks to form a final product).

By “polyketide” is meant a compound comprising multiple ketyl→ketide? units.

By “non-ribosomal peptide synthetase” or “non-ribosomal peptide synthase” or “NRPS” is meant a polypeptide or series of interacting polypetides that produce a non-ribosomal peptide, thus that is able to catalyze peptide bond formation without the presence of ribosomal components.

By “polyketide synthase” (PKS) is meant a polypeptide or series of polypeptides that produce a polyketide. By “alter an amount” is meant to change the amount, by either in-creasing or decreasing. An increase or decrease may be by 3%, 5%, 8%, 10%, 15%, 20%, 30%, 40%, 50%, 60%, 70%, 80%, 90%, 100%, or more.

By “non-ribosomal peptide synthetase/polyketide synthase hybrid” or hybrid of nonribosomal peptide synthetase and polyketide synthase” or “NRPS/PKS hybrid” or “hybrid of NRPS and PKS” or “hybrid of PKS and NRPS” is meant a enzyme systems comprising any domains or modules from non-ribosomal peptide synthetases and polyketide synthases resulting in the respective hybrid natural products.

By “altering a structure” any change in a chemical (e.g., covalent or noncovalent) bond as compared to a reference structure is meant.

By “mutation” an alteration in the nucleic acid sequence such that the amino acid sequence encoded by the nucleic acid sequence has at least one amino acid alteration from a naturally occurring sequence is meant. The mutation may, without limitation, be an insertion, deletion, frameshift mutation, or a missense mutation. This term also de-scribes a protein encoded by the mutant nucleic acid sequence.

By “variant” a polypeptide or polynucleotide with at least 10%, 20%, 30%, 40%, 50%, 60%, 70%, 80%, 90%, 95%, or 99% sequence identity to a reference sequence is meant. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications by applying substitution/scoring matrices (e.g. PAM, Blosum, GONET, JTT). Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-150 indicating a closely related se-quence (Altschul et al., 1990).

In a first aspect, the invention pertains to a chimeric protein for the production of nonribosomal peptides, comprising at least one chimeric thiolation domain (T-domain (PKS acyl carrier protein (ACP) or NRPS petidyl carrier (PCP))) consisting of a first T-domain (PKS acyl carrier protein (ACP) or NRPS petidyl carrier (PCP)) segment directly adjacent to a second T-domain (PKS acyl carrier protein (ACP) or NRPS petidyl carrier (PCP)) segment, wherein the first T-domain (PKS acyl carrier protein (ACP) or NRPS petidyl carrier (PCP)) segment comprises an amino acid sequence from an N-terminal part of a T-domain (PKS acyl carrier protein (ACP) or NRPS petidyl carrier (PCP)) amino acid sequence, and wherein the second T-domain (PKS acyl carrier protein (ACP) or NRPS petidyl carrier (PCP)) segment comprises an amino acid sequence from a C-terminal part of a T-domain (PKS acyl carrier protein (ACP) or NRPS petidyl carrier (PCP)) amino sequence; wherein the chimeric thiolation domain is a fully functional T-domain (PKS acyl carrier protein (ACP) or NRPS petidyl carrier (PCP)); characterized in that the amino acid sequences of the parts of the T-domain (PKS acyl carrier protein (ACP) or NRPS petidyl carrier (PCP)) amino acid sequences of the first and the second T-domain (PKS acyl carrier protein (ACP) or NRPS petidyl carrier (PCP)) segments are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.

In an alternative first aspect, the invention further pertains to a method for producing a chimeric non-ribosomal peptide synthetase (NRPS)- and/or polyketide (PKS)-module, the method comprising:
(a) Providing a first NRPS/PKS module sequence comprising at least a first T-domain (T-domain (PKS acyl carrier protein (ACP) or NRPS petidyl carrier (PCP)) sequence;
(b) Providing a second NRPS/PKS module sequence comprising at least a second T-domain sequence;
(c) Optionally, aligning the first T-domain sequence to the second T-domain sequence to identify at least one T-domain fusion point;
(d) Fusing by cloning directly adjacent to each other into a genetic construct a first partial sequence to a second partial sequence to obtain a chimeric module sequence, wherein
   (i) The first partial sequence is a nucleotide sequence encoding an amino acid sequence of the first NRPS/PKS module sequence located directly N-terminal to the identified T-domain fusion point; and
   (ii) The second partial sequence is a nucleotide sequence encoding an amino acid sequence of the second NRPS/PKS module sequence located directly C-terminal to the identified T-domain fusion point;
      and thereby producing a genetic construct comprising a chimeric NRPS- and/or PKS-module.

The invention is based on the surprising expoitation of a highly conserved sites within the T domain based on an evolutionary analysis of more than 225 aligned amino acid sequences of NRPS A-T-C tri-domains various species, including Photorhabdus and Xenorhabdus species, as well as representative NRPS of firmicutes, actinomycetes, cyanobacteria and other proteobacteria. This analysis of the invention identified the conserved FFxxGGxS motif in the T domain which forms the basis for generating chimeric proteins of the invention. This sequence motifs in NRPS sequences in T-domains are surprisingly effective as fusion points to combine NRPS sequences, which are naturally not combined in one single gene (therefore such combinations are chimeric/artificial).

It is a preferred embodiment of the invention that the first T-domain (PKS acyl carrier protein (ACP) or NRPS petidyl carrier (PCP)) segment is directly adjacent to, and connected with, the N-terminal end of the second T-domain (PKS acyl carrier protein (ACP) or NRPS petidyl carrier (PCP)) segment.

In another preferred embodiment, the chimeric protein is a chimeric non-ribosomal peptide synthetases (NRPSs), a chimeric polyketide synthase (PKSs), or a chimeric NRPS/PKS hybrid.

In accordance with the invention it may be further preferred that the chimeric protein of the invention further comprises one or more known NRPS and/or PKS domains, in particular any or any combination of Adenylation (A) domain(s), Condensation (C) domain(s), Epimerization (E) domain(s), Reductase (R) domain(s), C/E domain(s), Oxidation (Ox) domain(s), Reduktion (Red) domain(s), hetero cyclisation (Cy) domain(s), starter C domain(s), Methylation (Mt) domain(s), A-Mt domain(s), A-Ox domain(s), Communication (Com) domain(s), Formylation (F) domain(s), X (X) domains, and/or terminal thioesterase (TE) domain(s) or terminal C domains; and/or the PKS domains Acyltransferase (AT) domain(s), Ketosynthase (KS) domain(s), Dehydratase (DH) domain(s), Ketoreductase (KR) domain(s), Enoylreductase (ER) domain(s), Methyltransferase (Mt) O- or C-domain(s), Docking (DD) domain(s), Thioesterase (TE) domain(s), PLP-dependent cysteine lyase (SH) domain(s), Acylcarrierprotein (ACP). NRPS, fungal and bacterial, or related domains and domain structures are well known to the skilled artisan.

In another embodiment it is preferably that the chimeric T domain consists of the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence.

A chimeric protein in accordance with the invention is preferred, wherein the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence are fused together at a fusion site, and wherein said fusion site is located+/−5 amino acids around the fusion sites 1 to 7 as indicated in FIG. 3 herein, preferably the around fusion sites 3 and 4.

One further embodiment of the invention is directed at a chimeric protein, wherein the fusion site is directly before or after any amino acid position of the conserved T domain motif-FFxxGG (H/N/D)S-, wherein x is any amino acid.

In an additional embodiment in section (a) of the first aspect, the protein further comprises at least one NRPS/PKS domain N-terminal of the chimeric T domain derived from a first microorganism species, and at least one NRPS/PKS domain C-terminal of the chimeric T domain and which is derived from a second microorganism species, with the provision that the first and the second microorganism species are not identical.

Another preferred embodiment is that the chimeric protein comprises a sequence of NRPS/PKS domains that is capable of catalysing peptide and/or hybrid peptide-polyketide synthesis.

It is a preferred embodiment, for example in context with the first alternative aspect, wherein the first NRPS/PKS module sequence and the second NRPS/PKS module sequence are not identical, preferably wherein the first T-domain sequence and the second T-domain sequence are not identical.

It is another preferred embodiment, for example in context with the first alternative aspect, wherein the first NRPS/PKS module sequence and the second NRPS/PKS module sequence are derived from the same NRPS/PKS gene, but are not directly adjacent to each other.

It is yet another preferred embodiment, for example in context with the first alternative aspect, wherein the first NRPS/PKS module sequence and the second NRPS/PKS module sequence are derived from two different NRPS/PKS genes; preferably of two different bacterial and/or fungal species.

In context with for example the first alternative aspect, it may be preferably, that the T-domain comprises both a T-domain linker sequence and T-domain protein domain sequence.

In context with for example the first alternative aspect, it may be preferably, that the T-domain fusion point is selected to be positioned at one of the following positions of the aligned sequences with reference to the T-domain structure comprising from N- to C-terminus: T-domain linker domain, Helix-1, Loop-1, Helix-2, Loop-2, Helix-3, Helix-4, wherein:
(a) Within or N-terminal of the T-domain linker domain,
(b) Within the Loop-1 domain;
(c) Within the Helix-2;
(d) C-terminal of the Helix-4.

In the first alternative aspect, the method further comprises expressing the chimeric NRPS- and/or PKS-module from the genetic construct.

In a second aspect, the invention pertains a system for the generation of chimeric nonribosomal peptide synthetases (NRPSs), a chimeric polyketide synthase (PKSs), or a chimeric NRPS/PKS hybrid, comprising at least two system units:
(a) a first system unit comprising at least the first T-domain (PKS acyl carrier protein (ACP) or NRPS petidyl carrier (PCP)) segment recited in any one of the other aspects and embodiments of this aspects, and
(b) a second system unit comprising at least the second T domain segment recited in any one of the other aspects and embodiments of this aspects;
   wherein each system unit may comprise one or more of further NRPS/PKS domains, and wherein the chimeric non-ribosomal peptide synthetases (NRPSs), the chimeric polyketide synthase (PKSs), or the chimeric NRPS/PKS hybrid is obtainable by fusing the first and the second system unit such that the N-terminal T-domain (PKS acyl carrier protein (ACP) or NRPS petidyl carrier (PCP)) amino acid sequence of the first system unit is directly fused to the C-terminal T-domain (PKS acyl carrier protein (ACP) or NRPS petidyl carrier (PCP)) amino acid sequence of the second system unit.

In a preferred embodiment, the sequences of the first system unit and the second system unit comprise amino acid sequences of the parts of the T-domain (PKS acyl carrier protein (ACP) or NRPS petidyl carrier (PCP)) amino sequences of the first and the second T-domain (PKS acyl carrier protein (ACP) or NRPS petidyl carrier (PCP)) segments, which are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.

In a third aspect, the invention pertains to a method for the generation of chimeric nonribosomal peptide synthetases (NRPSs), a chimeric polyketide synthase (PKSs), or a chimeric NRPS/PKS hybrid, the method comprising the steps of:
(a) Providing a first NRPS or PKS gene sequence from a first microorganism species,
(b) Providing a second NRPS or PKS gene sequence from a second microorganism species, and
(c) Combining at least one part of the first NRPS or PKS gene sequence with at least one part of the second NRPS or PKS gene sequence to obtain a chimeric gene sequence by fusing both sequences to each other such that at least one fusion point is located within a T domain, and wherein the fusion product comprises a T domain consisting of an N-terminal sequence of a T domain of the first NRPS or PKS gene sequence and a C-terminal sequence of a T domain of the second NRPS or PKS gene sequence, and
(d) Optionally, expressing the chimeric gene sequence to obtain the chimeric nonribosomal peptide synthetases (NRPSs), a chimeric polyketide synthase (PKSs), or a chimeric NRPS/PKS hybrid.

In a fourth aspect, the invention pertains to a method for combining, or producing a combination of, at least two NRPS/PKS modules from two different NRPS/PKS genes or from two separated locations within the same NRPS/PKS gene, the method comprising the steps of:
(a) Identifying a first target NRPS/PKS module that is to be combined directly N-terminally to a second target NRPS/PKS module;
(b) Identifying a second target NRPS/PKS module that is to be combined directly C-terminally to a second target NRPS/PKS module, and wherein the second target NRPS/PKS module is selected from a module that is located within a NRPS/PKS gene C-terminally to a third non-target NRPS/PKS module;
(c) Combining the first target NRPS/PKS module with the second target NRPS/PKS module by directly attaching an N-terminal part of the T domain of the first NRPS/PKS module with a C-terminal part of a T domain of the third non-target NRPS/PKS module;
   Wherein the combination of the N-terminal part of the T domain of the first NRPS/PKS module with the C-terminal part of a T domain of the third non-target NRPS/PKS module constitute a full and functional T-domain (PKS acyl carrier protein (ACP) or NRPS petidyl carrier (PCP)).

In a preferred embodiment of the invention, the first and the third NRPS/PKS modules each comprise at least a thiolation domain.

It is also preferably that the combining may involve one or more further NRPS/PKS modules and/or domains N-terminally of the first target NRPS/PKS module.

Alternatively or additionally, the combining may involve one or more further NRPS/PKS modules and/or domains C-terminally of the second target NRPS/PKS module.

A preferred embodiment further pertains to the first target NRPS/PKS module and the third non-target NRPS/PKS module are not identical.

In a fifth aspect, the invention pertains to a combination product obtained by the method of the fourth aspect.

In a sixth aspect, the invention pertains to a method for the generation of a nucleic acid construct encoding a system unit recited in the other aspects and embodiments of this aspects, the method comprising a step of amplifying a sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S, wherein x is any amino acid.

In a seventh aspect, the invention pertains to a method for generating a library of nucleic acid constructs each encoding a system unit recited of the other aspects and embodiments of this aspects, wherein the method comprises a step of amplifying a first sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S; and wherein the method comprises a second amplification of at least a second sequence stretch of an NRPS/PKS gene using the degenerate primer pair from the same or one or more different NRPS/PKS genes.

In an eighth aspect, the invention pertains to a system of genetic constructs for the expression of a chimeric NRPS/PKS, wherein the system comprises at least one genetic construct comprising a chimeric NRPS- and/or PKS-module as produced by a method of the previous aspects.

In particular, certain aspects as described are preferred in the following embodiments and items:
Item 1. A chimeric protein for the production of non-ribosomal peptides, comprising at least one chimeric thiolation domain (T-domain (PKS acyl carrier protein (ACP) or NRPS petidyl carrier (PCP))) consisting of a first T-domain (PKS acyl carrier protein (ACP) or NRPS petidyl carrier (PCP)) segment directly adjacent to a second T-domain (PKS acyl carrier protein (ACP) or NRPS petidyl carrier (PCP)) segment, wherein the first T-domain (PKS acyl carrier protein (ACP) or NRPS petidyl carrier (PCP)) segment comprises an amino acid sequence from an N-terminal part of a T-domain (PKS acyl carrier protein (ACP) or NRPS petidyl carrier (PCP)) amino acid sequence, and wherein the second T-domain (PKS acyl carrier protein (ACP) or NRPS petidyl carrier (PCP)) segment comprises an amino acid sequence from a C-terminal part of a T-domain (PKS acyl carrier protein (ACP) or NRPS petidyl carrier (PCP)) amino sequence; wherein the chimeric thiolation domain is a fully functional T-domain (PKS acyl carrier protein (ACP) or NRPS petidyl carrier (PCP)); characterized in that
the amino acid sequences of the parts of the T-domain (PKS acyl carrier protein (ACP) or NRPS petidyl carrier (PCP)) amino sequences of the first and the second T-domain (PKS acyl carrier protein (ACP) or NRPS petidyl carrier (PCP)) segments are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.
Item 2. The chimeric protein of item 1, wherein the first T-domain (PKS acyl carrier protein (ACP) or NRPS petidyl carrier (PCP)) segment is directly adjacent to, and connected with, the N-terminal end of the second T-domain (PKS acyl carrier protein (ACP) or NRPS petidyl carrier (PCP)) segment.
Item 3. The chimeric protein of item 1 or 2, wherein the chimeric protein is a chimeric nonribosomal peptide synthetases (NRPSs), a chimeric polyketide synthase (PKSs), or a chimeric NRPS/PKS hybrid.
Item 4. The chimeric protein of any one of items 1 to 3, further comprising one or more known NRPS and/or PKS domains, in particular any or any combination of Adenylation (A) domain(s), Condensation (C) domain(s), Epimerization (E) domain(s), Reductase (R) domain(s), C/E domain(s), Oxidation (Ox) domain(s), Reduktion (Red) domain(s), hetero cyclisation (Cy) domain(s), starter C domain(s), Methylation (Mt) domain(s), A-Mt domain(s), A-Ox domain(s), Communication (Com) domain(s), Formylation (F) domain(s), X (X) domains, and/or terminal thioesterase (TE) domain(s) or terminal C domains; and/or the PKS domains Acyltransferase (AT) domain(s), Ketosynthase (KS) domain(s), Dehydratase (DH) domain(s), Ketoreductase (KR) domain(s), Enoylreductase (ER) domain(s), Methyltransferase (Mt) O- or C-domain(s), Docking (DD) domain(s), Thioesterase (TE) domain(s), PLP-dependent cysteine lyase (SH) domain(s), Acylcarrierprotein (ACP)
Item 5. The chimeric protein of any one of items 1 to 4, wherein the chimeric T domain consists of the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence.
Item 6. The chimeric protein of any one of items 1 to 5, wherein the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence are fused together at a fusion site, and wherein said fusion site is located+/−5 amino acids around the fusion sites 1 to 7 indicated in FIG. 3, preferably fusion sites 3 and 4.
Item 7. The chimeric protein of item 6, wherein the fusion site is directly before or after any amino acid position of the conserved T domain motif-FFxxGG(H/N/D)S-, wherein x is any amino acid.
Item 8. The chimeric protein of any one of items 1 to 7, wherein in item 1 (a) the protein further comprises at least one NRPS/PKS domain N-terminal of the chimeric T domain derived from a first microorganism species, and at least one NRPS/PKS domain C-terminal of the chimeric T domain and which is derived from a second microorganism species, with the provision that the first and the second microorganism species are not identical.
Item 9. The chimeric protein of any one of items 1 to 8, wherein the chimeric protein comprises a sequence of NRPS/PKS domains that is capable of catalysing peptide and/or hybrid peptide-polyketide synthesis.
Item 10. A system for the generation of chimeric non-ribosomal peptide synthetases (NRPSs), a chimeric polyketide synthase (PKSs), or a chimeric NRPS/PKS hybrid, comprising at least two system units:
(a) a first system unit comprising at least the first T-domain (PKS acyl carrier protein (ACP) or NRPS petidyl carrier (PCP)) segment recited in any one of items 1 to 9, and
(b) a second system unit comprising at least the second T domain segment recited in any one of items 1 to 9;
wherein each system unit may comprise one or more of further NRPS/PKS domains, and wherein the chimeric non-ribosomal peptide synthetases (NRPSs), the chimeric polyketide synthase (PKSs), or the chimeric NRPS/PKS hybrid is obtainable by fusing the first and the second system unit such that the N-terminal T-domain (PKS acyl carrier protein (ACP) or NRPS petidyl carrier (PCP)) amino acid sequence of the first system unit is directly fused to the C-terminal T-domain (PKS acyl carrier protein (ACP) or NRPS petidyl carrier (PCP)) amino acid sequence of the second system unit.
Item 11. The system of item 10, wherein the sequences of the first system unit and the second system unit comprise amino acid sequences of the parts of the T-domain (PKS acyl carrier protein (ACP) or NRPS petidyl carrier (PCP)) amino sequences of the first and the second T-domain (PKS acyl carrier protein (ACP) or NRPS petidyl carrier (PCP)) segments, which are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.
Item 12. A method for the generation of chimeric non-ribosomal peptide synthetases (NRPSs), a chimeric polyketide synthase (PKSs), or a chimeric NRPS/PKS hybrid, the method comprising the steps of:
(a) Providing a first NRPS or PKS gene sequence from a first microorganism species,
(b) Providing a second NRPS or PKS gene sequence from a second microorganism species,
(c) Combining at least one part of the first NRPS or PKS gene sequence with at least one part of the second NRPS or PKS gene sequence to obtain a chimeric gene sequence by fusing both sequences to each other such that at least one fusion point is located within a T domain, and wherein the fusion product comprises a T domain consisting of an N-terminal sequence of a T domain of the first NRPS or PKS gene sequence and a C-terminal sequence of a T domain of the second NRPS or PKS gene sequence, and
(d) Optionally, expressing the chimeric gene sequence to obtain the chimeric non-ribosomal peptide synthetases (NRPSs), a chimeric polyketide synthase (PKSs), or a chimeric NRPS/PKS hybrid.
Item 13. A method for combining, or producing a combination of, at least two NRPS/PKS modules from two different NRPS/PKS genes or from two separated locations within the same NRPS/PKS gene, the method comprising the steps of:
(a) Identifying a first target NRPS/PKS module that is to be combined directly N-terminally to a second target NRPS/PKS module;
(b) Identifying a second target NRPS/PKS module that is to be combined directly C-terminally to a second target NRPS/PKS module, and wherein the second target NRPS/PKS module is selected from a module that is located within a NRPS/PKS gene C-terminally to a third non-target NRPS/PKS module;
(c) Combining the first target NRPS/PKS module with the second target NRPS/PKS module by directly attaching an N-terminal part of the T domain of the first NRPS/PKS module with a C-terminal part of a T domain of the third non-target NRPS/PKS module;

Wherein the combination of the N-terminal part of the T domain of the first NRPS/PKS module with the C-terminal part of a T domain of the third non-target NRPS/PKS module constitute a full and functional T-domain (PKS acyl carrier protein (ACP) or NRPS petidyl carrier (PCP)).
Item 14. The method of item 13, wherein the first and the third NRPS/PKS modules each comprise at least a thiolation domain.
Item 15. The method of item 13 or 14, wherein the combining may involve one or more further NRPS/PKS modules and/or domains N-terminally of the first target NRPS/PKS module.
Item 16. The method of any one of items 13 to 15, wherein the combining may involve one or more further NRPS/PKS modules and/or domains C-terminally of the second target NRPS/PKS module.
Item 17. The method of any one of items 13 to 15, wherein the first target NRPS/PKS module and the third non-target NRPS/PKS module are not identical.
Item 18. A combination product obtained by the method of any one of items 13 to 17.
Item 19. A method for the generation of a nucleic acid construct encoding a system unit recited in item 10, the method comprising a step of amplifying a sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S, wherein x is any amino acid.
Item 20. A method for generating a library of nucleic acid constructs each encoding a system unit recited in item 10, wherein the method comprises a step of amplifying a first sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S; and
wherein the method comprises a second amplification of at least a second sequence stretch of an NRPS/PKS gene using the degenerate primer pair from the same or one or more different NRPS/PKS genes.
Item 21. A method for producing a chimeric non-ribosomal peptide synthetase (NRPS) and/or polyketide (PKS)-module, the method comprising:
(a) Providing a first NRPS/PKS module sequence comprising at least a first T-domain (T-domain (PKS acyl carrier protein (ACP) or NRPS petidyl carrier (PCP)) sequence;
(b) Providing a second NRPS/PKS module sequence comprising at least a second T-domain sequence;
(c) Optionally, aligning the first T-domain sequence to the second T-domain sequence to identify at least one T-domain fusion point;
(d) Fusing by cloning directly adjacent to each other into a genetic construct a first partial sequence to a second partial sequence to obtain a chimeric module sequence, wherein
i. The first partial sequence is a nucleotide sequence encoding an amino acid sequence of the first NRPS/PKS module sequence located directly N-terminal to the identified T-domain fusion point; and
ii. The second partial sequence is a nucleotide sequence encoding an amino acid sequence of the second NRPS/PKS module sequence located directly C-terminal to the identified T-domain fusion point;
and thereby producing a genetic construct comprising a chimeric NRPS- and/or PKS-module.
Item 22. The method of item 21, wherein the first NRPS/PKS module sequence and the second NRPS/PKS module sequence are not identical, preferably wherein the first T-domain sequence and the second T-domain sequence are not identical.
Item 23. The method of item 21 or 22, wherein the first NRPS/PKS module sequence and the second NRPS/PKS module sequence are derived from the same NRPS/PKS gene, but are not directly adjacent to each other.
Item 24. The method of item 21 or 22, wherein the first NRPS/PKS module sequence and the second NRPS/PKS module sequence are derived from two different NRPS/PKS genes; preferably of two different bacterial and/or fungal species.
Item 25. The method of any one of items 21 to 24, wherein the T-domain comprises both a T-domain linker sequence and T-domain protein domain sequence.
Item 26. The method of any one of items 21 to 25, wherein the T-domain fusion point is selected to be positioned at one of the following positions of the aligned sequences with reference to the T-domain structure comprising from N- to C-terminus: T-domain linker domain, Helix-1, Loop-1, Helix-2, Loop-2, Helix-3, Helix-4, wherein
(a) Within or N-terminal of the T-domain linker domain,
(b) Within the Loop-1 domain;
(c) Within the Helix-2;
(d)C-terminal of the Helix-4.
Item 27. The method of any one of items 21 to 26, further comprising expressing the chimeric NRPS- and/or PKS-module from the genetic construct.
Item 28. A system of genetic constructs for the expression of a chimeric NRPS/PKS, wherein the system comprises at least one genetic construct comprising a chimeric NRPS- and/or PKS-module as produced by any one of items 21 to 27.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: Phosphopantetheinylation of the apo-T (PCP) domain to holo enzyme by Sfp. Top: Crystal structure of Sfp with its substrate coenzyme A and Mg2+ (PDB-ID: 1QR0)³⁷. Centre: The phosphopantetheine moiety of coenzyme A (red) is covalently attached to a conserved serine residue of the T (PCP) domain by Sfp. Bottom: Crystal structure of a T (PCP) domain, solved by Weber et al. (PDB-ID: 1DNY)³⁸.

FIG. 2: Overview of the applied in silico workflow. To get a better understanding of NRPS evolution as well as to detect evolutionary hot-spots of recombination NRPSs from Photo- and Xenorhabdus sp. were analysed in silico. Initially a fine-grained phylogenetic reconstruction analysis was performed (a) along with tree-topological analysis (b) by applying the software DualBrothers²⁴. Gained results indicated that different parts of T domains show different topologies, indicating different evolutionary origins. Next, the recombination detection program RDP4²⁵ was applied to detect potential evolutionary hot-spots (c). RDP4 frequently suggested that homologous recombination appeared either within A domains or directly in front of T domains as well as close to the invariant Serine. Further nucleotide sequence analysis revealed the presence of an AT-skew in the conserved area directly in front (5′) of the invariant Serine, theoretically ideally suited to promote homologous recombination. Further phylogenetic reconstructions (d) of T domains split at the invariant Serine into a N- and C-terminal subunit, showed divergent clustering from each other, indicating that in the course of evolution homologous recombination may be more common within T domains than in front of them. Taken together a, b, c, and d lead us to the evolutionary hypothesis depicted in (e).

FIG. 3: Recombination point screening to detect functional splicing positions within T domains. To analyse if T domains can be targeted to functionally recombine NRPS building blocks Cstart-A1-Tp1 of Xenoamicin⁷² producing NRPS from X. stockiae was fused with pT3-C/E4-A4-T4-C/E5-A5-T5-TE of the GameXPetptide producing NRPS GxpS. Splicing positions 1, 3, and 4 showed production of the expected lipopeptide. Top: Schematic representation of the resulting chimeric NRPS (left), and the lipopeptide's chemical structure (right). Middle: Ribbon representation of the crystallised T domain (left) of EntF (PDB: 4ZXJ) with tested fusion points (1-7) highlighted in black. Fusion points resulting in catalytically active NRPSs are marked with a red arrow. EICs of m/z [M+H]⁺=444.3 produced in E. coli DH10B::mtaA are shown on the right. The numbers refer to the individual recombinant NRPSs (1-7). Bottom: Sequence alignment of targeted T domains. Functional recombination sites are highlighted red. Centre: Depicted is the colour code of the NRPS buildings blocks used. For domain assignment the following symbols are used: A, adenylation domain, large circles; T, thiolation domain, rectangle; C, condensation domain, triangle; C/E, dual condensation/epimerization domain, diamond; TE, thioesterase domain, small circle.

FIG. 4: Proof of Concept—Fusion Site 3. To validate if the evolutionary inspired novel fusion site 3 can be applied to reprogram NRPS in general NRPS-1 to -3 were created. Top: Schematic representation of recombinant NRPS-1-3 as well as associated products. Middle: Depicted is the colour code of the NRPS buildings blocks used. For domain assignment the following symbols are used: A, adenylation domain, large circles; T, thiolation domain, rectangle; C, condensation domain, triangle; C/E, dual condensation/epimerization domain, diamond; TE, thioesterase domain, small circle, R, Reductase domain, square, and the used synthetic zipper (SZ) pair, SZ17: 18. Bottom: Structures of peptides 1-12 produced from NRPS-1 to -3 expressed in E. coli DH10B::mtaA

FIG. 5: shows De novo design of chimeric NRPSs to produce Lipopeptide-Aldehydes. To verify if the evolutionary inspired novel fusion site 3 can be applied to de novo design NRPS from scratch NRPS-4 to -8 were created. Top: Schematic representation of recombinant NRPS4-8 as well as associated products. Middle: Depicted is the colour code of the NRPS buildings blocks used. For domain assignment see FIG. 4. Bottom: Structures of peptides 13-17 produced from NRPS-4 to -8 expressed in E. coli DH10B::mtaA

FIG. 6: Turning NRPS into artificial NRPS/PKS hybrid synthetase (part 1). Top: Schematic representation of recombinant NRPS-PKS-9 as well as associated products. Middle: Depicted is the colour code of the NRPS and PKS buildings blocks used. For domain assignment see FIG. 4; further symbols: KS, DH, and KR domains, small circles. Bottom: Structures of 18-23 produced from NRPS-PKS-9 expressed in E. coli DH10B::mtaA.

FIG. 7: Turning NRPS into artificial NRPS/PKS hybrid synthetase (part 2). Top: Schematic representation of recombinant PKS-NRPS-10. Middle: Depicted is the colour code of the NRPS and PKS buildings blocks used. For domain assignment see FIG. 4 and FIG. 6. Bottom: Structure of 24 produced from PKS-NRPS-10 expressed in E. coli DH10B::mtaA.

FIG. 8: Sequence alignment of selected T domains. To verify and compare fusion points 1 and 4 a series of recombinant NRPSs was created (See FIGS. 9-11). Highlighted with arrows are the applied recombination sites to create NRPS-11 to -24

FIG. 9: Recombining non-related building blocks of GC-content and codon usage to create chimeric NRPS—(part 1). To verify and compare the applicability of fusion points 1 and 4 a series of recombinant NRPS was created; Fusion point 1: NRPS-11 and -13; Fusion point 4: NRPS-12 and -14. Top: Schematic representation of the unpublished WT-NRPS as well as recombinant NRPS-11 to -14. Middle: Depicted is the colour code of the NRPS buildings blocks used. For domain assignment see FIG. 4. Bottom: Structures of 25-27 produced from NRPS-11 to -14 expressed in E. coli DH10B::mtaA

FIG. 10: Recombining non-related building blocks of GC-content and codon usage to create chimeric NRPS—(part 2). To verify and compare the applicability of fusion points 1 and 4 a series of recombinant NRPS was created; Fusion point 1: NRPS-15 and -17; Fusion point 4: NRPS-16 and -18. Top: Schematic representation of recombinant NRPS-15 to -19. Middle: Depicted is the colour code of the NRPS buildings blocks used. For domain assignment see FIG. 4. Bottom: Structures of 28 and 29 produced from NRPS-15 to -18 expressed in E. coli DH10B::mtaA.

FIG. 11: Recombining non-related building blocks of varying GC-content and codon usage to create chimeric NRPS—(part 3). To verify and compare the applicability of fusion points 1 and 4 a series of recombinant NRPS was created; Fusion point 1: NRPS-19, and -21; Fusion point 4: NRPS-20, and -22. Top: Schematic representation of the recombinant NRPS-19 to -22. Middle: Depicted is the colour code of the NRPS buildings blocks used. For domain assignment see FIG. 4. Bottom: Structure of 30 produced from NRPS-19 to -22 expressed in E. coli DH10B::mtaA.

FIG. 12: Scheme of T-Domain Degenerated Primer Library. Steps A to G are described in the Examples.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### Background: Thiolation Domains (T Domain)

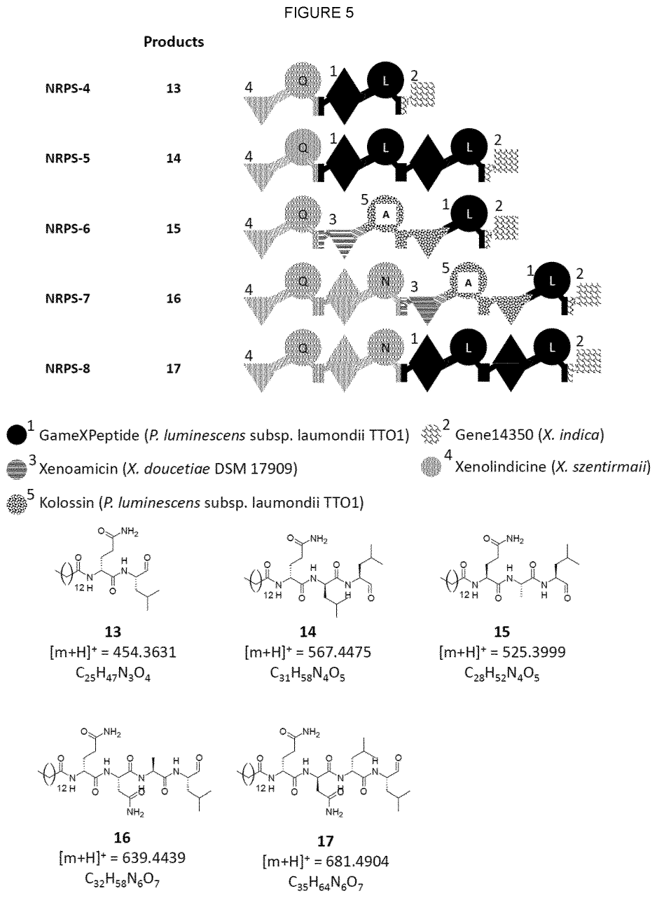

The T domain, is the only NRPS domain without an autonomous catalytic activity and is responsible for transportation of substrates and elongation intermediates to the catalytic centres26. The ~100 AA long protein belongs to the acyl-carrier protein (ACP-) like superfamily and is located C-terminally of the A domain and N-terminally of the C domain (FIG. 5B).

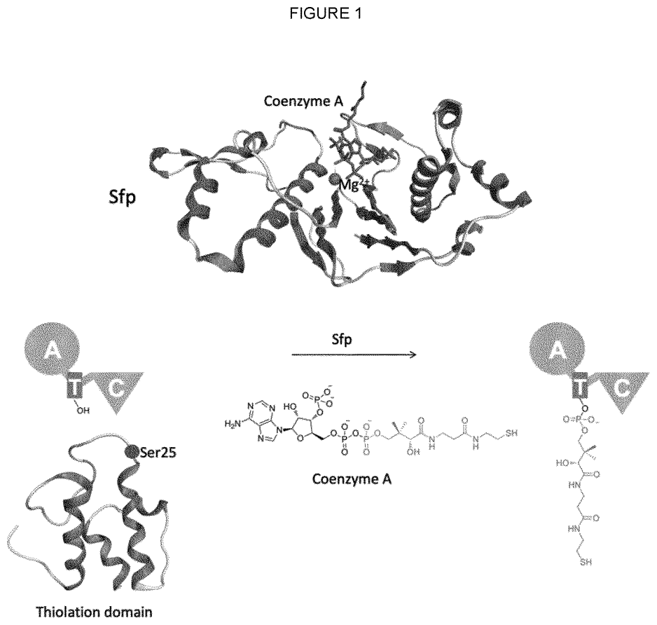

The A domain catalyses two half reactions27. In the second half reaction the activated (adenylated) substrate is transferred onto the terminal cysteamine thiol group of the 4′Ppan cofactor covalently attached to the T domain. The T domain together with its 4′Ppan cofactor represents the holo-protein, which is derived from the apo-carrier protein by post-translational modification28. The ~20 Å long prosthetic group of coenzyme A (CoA) derived phosphopantetheinyl, bearing the terminal thiol, is covalently connected via a phosphodiester to the sidechain of a strictly conserved serine surrounded by a homologous pattern of AA residues of the folded apo-carrier protein29-33. Transfer of 4′Ppan onto the apo-protein is achieved by action of NRPS-specialized 4′-phosphopantetheinyl transferases (PPTase), often encoded as part of a NRPS biosynthetic gene cluster (FIG. 1) (e.g., Sfp from B. subtilis) 34-37.

The first T domain structure was solved by Weber and colleagues using NMR spectroscopy with the T domain of the third module of the B. brevis tyrocidine synthetase (FIG. 1) 38. Weber et al. revealed that the T domain is a distorted four-helix bundle with an extended loop between the first two helices and that the invariant serine residue, the site of cofactor binding, is located at the interface between this loop and the second helix. Furthermore, no binding pocket can be found within the T domain, which agrees with the lack of substrate selectivity. Furthermore, the prosthetic group shows no interaction with the protein and is accommodated in the solvent. With respect to functionality and structural fold, T domains are similar to acyl carrier proteins (ACP) from fatty acid synthases and PKS26. Yet, sequence homologies between them only exist in the immediate neighbourhood of the invariant serine residue39-41. The most obvious difference between them is the overall charge of the proteins. ACPs have predominantly acidic side chains on their surface, while T domains are less polar. This finding corresponds to the charges of the associated 4′Ppan-transferases (AcpS and Sfp like PPtases). Further, crystal structures of carrier proteins—as excised domains, free standing single proteins, and during recognition by PPtase like Sfp from B. subtilis and AcpS from Streptomyces coelicolor or Streptococcus pneumoniae—revealed that Sfp has one V-shaped interface for the 4′Ppan cofactor modification of a T domain, whereas AcpS has three catalytic sites for post-translational modification28,38,39,42-50.

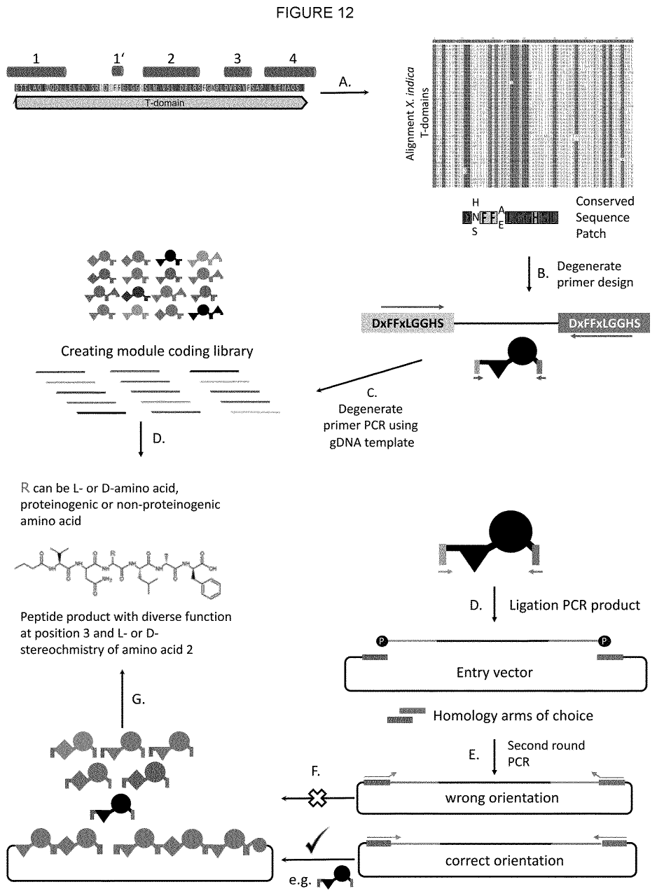

An A-T didomain is defined as initiation module since both proteins are required to activate and covalently tether the first substrate for subsequent peptide assembly26. During nonribosomal synthesis T domains hand over reaction intermediates among different catalytic centres and in doing so adopt alternative conformations42. The crystal structure of the surfactin A-C (SrfAC) termination module (FIG. 12) solved by Tanovic and colleagues (2008) constituted a major contribution to understanding the different domain arrangements during a catalytic cycle51. In this structure, the PCP domain was positioned to interact with the condensation domain. However, the attachment site for the 4′Ppan arm of the T domain was located 60 and 45 Å from the active sites of the A and TE domains, respectively, suggesting that large conformational rearrangements were required to deliver the pantetheine thiol to the other domains. At least three functionally relevant and distinct orientations have to be adopted by the T domain. The first conformation permits aminoacyl transfer onto the thiol group of the 4′Ppan by association with the A domain. The two remaining orientations within an elongation module are the approach of the T domain to the catalytic centre of the previous C domain at its acceptor site, and to the catalytic centre of the following C domain at its donor site.

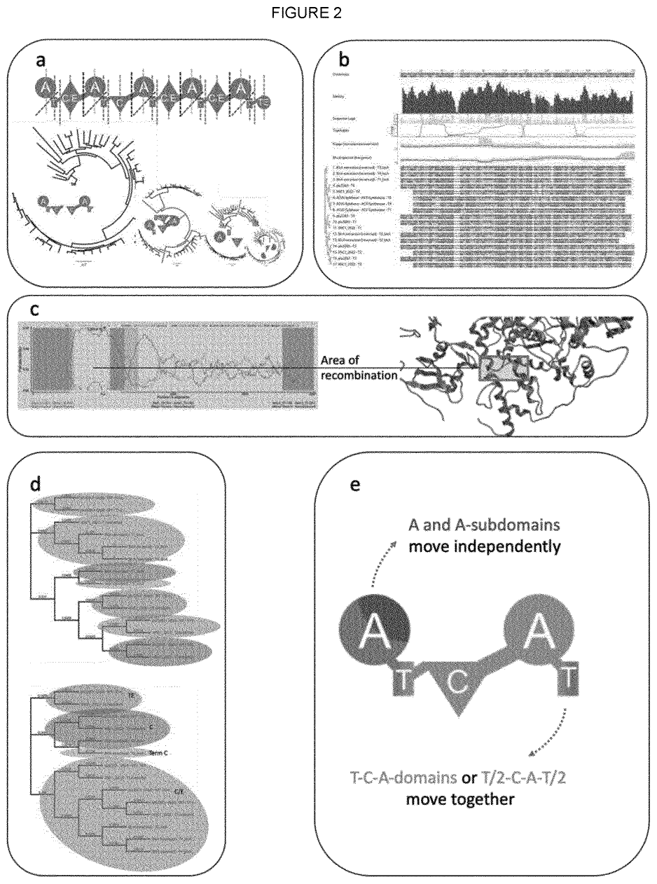

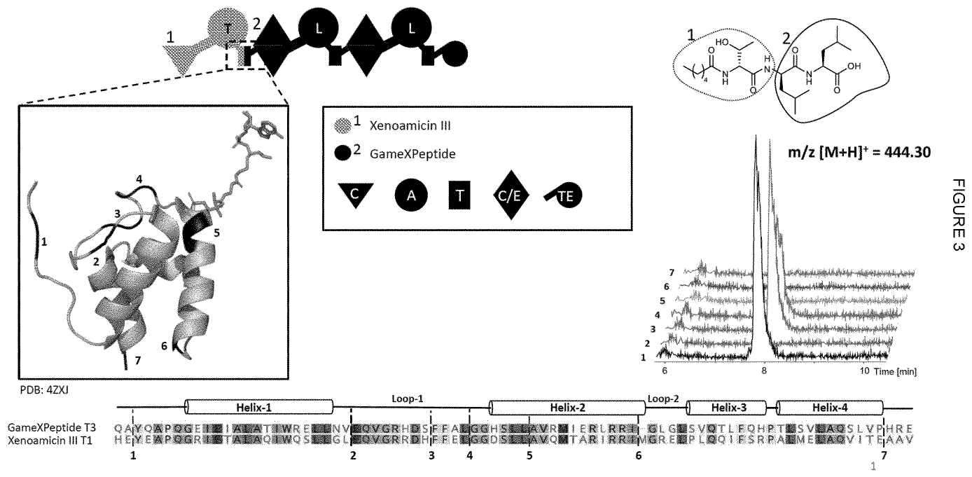

As evolutionary recombination analysis (FIG. 2) naturally does not come up with one specific splicing position but, with an area (sequence stretch) that is likely to promote homologous recombination, initially a fusion point screening was performed (FIG. 3)—to verify whether chimeric NRPS can be generated at all by targeting the T domain as well as to identify fusion sites resulting in the best peptide production. In brief, this screening led to the identification of three functional fusion sites (FIG. 3).

Especially fusion sites 3 and 4 are lying within an area that has some very interesting features (I-III):
(I) Firstly, they are in the immediate neighbourhood of the invariant serine residue that serves as 4′Ppan attachment site (FIG. 1), which is surrounded by a conserved pattern of AA residues (FFxxGG (H/D) S) and is homologous to the very area of PKS ACP domains.
(II) Secondly, on nucleotide level a further remarkable feature can be observed. The ~50 nucleotide spanning area not only is AT-rich and conserved among different unrelated bacterial species, even within GC-rich organisms like Actinobacteria, but, also shows an unequal frequency of the four DNA bases on both single strands of the respective DNA molecule. This mathematically unequal distribution is referred to as AT-skew52,53 and not only represents a statistical bias but, more importantly, indicates a genetic and codon stability that seems to resists mutations—i.e., transversions are statistically strongly underrepresented, thus the AT skew prevails and it is unlikely that point mutations change the AA encoded at this site.
(III) Thirdly, both T-domain parts, located upstream (N-terminal) and downstream (C-terminal) of the above-mentioned conserved AA stretch (FIG. 2d), do show a different clustering when phylogenetic reconstruction is performed. While the N-terminal part of the T-domains cluster like the respective upstream A-domains, the C-terminal part of the T-domains cluster like the neighbouring C domains. This behaviour also could be observed when doing phylogenetic reconstructions (FIG. 2a) along with tree-topological analysis (FIG. 2b) 24, indicating that this area represents a yet unnoticed potential evolutionary recombination site.

Taken together (I-III), these in silico observations (FIG. 2) along with the results from the in vivo conducted fusion-point screening (FIG. 3) lead us to the hypothesis that both, T-C-A units (fusion point 1), partial-T-C-A-T-partial (pT-C-A-Tp) units (fusion point 3 & 4), and combinations thereof ((1, 3), (1, 4), (3, 4), (4, 3), (4, 1), (3, 1)) may serve as ideal starting points to do evolutionary inspired megasynth(et)ase engineering (FIG. 2e).

The examples show:

### Example 1: Proof of Concept

This example serves as prove that NRPS elongation (NRPS-1) and starter units (NRPS2 & 3) functionally can be exchanged via fusion point 3 (FIG. 3).

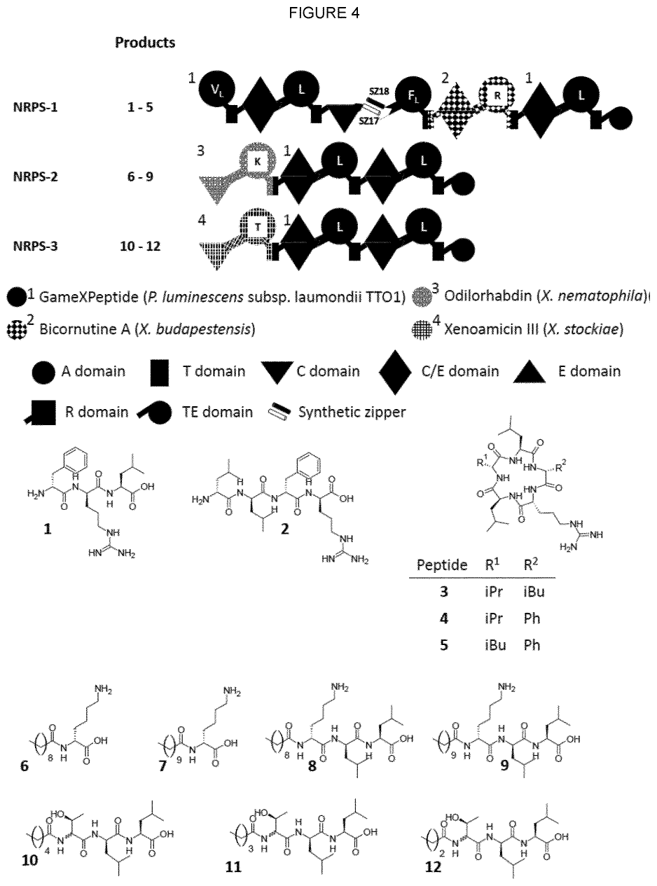

Prior to present work we created the typeS variant of the GameXPeptide (Gxp) A-E producing synthetase (GxpS)3,54. This artificial/synthetic two component NRPS (FIG. 4) was chosen to verify that fusion point 3 indeed can be applied to create new Gxp derivatives. By targeting the GxpS domains T3 and T4 it was possible to functionally exchange the Leucine specifying building block pT3-C4-A4-Tp4 of GxpS against the Arginine specifying building block pT3-C4-A4-Tp4 of the bicornutin producing NRPS (BicA) from X. budapestensis (FIG. 4). HPLCMS/MS analysis of methanolic culture extracts lead to the detection of five Arginine carrying peptides (1-5; FIG. 4). Whereas peptides 1 and 2 are the result of the unpaired activity of NRPS1 subunit 2, peptides 3-5 represent the expected full length Gxp derivatives. Due to the relaxed substrate specificities of A1 (Val, Leu) and A3 (Phe, Leu) 3-5 differ in position 1 and 3. Additionally, NRPS-2 and -3 were created by replacing GxpS A3-Tp3 of subunit 2 from typeS GxpS against two different initiation building blocks (Cstart-A-Tp) of the odilorhabdin55 and xenoamicin III56 synthesising NRPSs from X. nematophila HGB081 and X. stockiae, respectively (FIG. 4). Both NRPSs showed biosynthetic activity, synthesising lipopeptides 6-12. Whereas NRPS-3 only produced the expected lipo-tri-peptides (10-12), NRPS-2 also synthesised two C-terminally truncated versions of the lipo-tri-peptide 6.

### Example 2: De Novo Design of Chimeric NRPSs to Produce Lipopeptide-Aldehydes

This example (NRPS-4 to 8) serves to highlight how fusion point 3 can be leveraged to exchange termination units to create synthetic NRPSs with terminal Reductase (R) domains57,58 (FIG. 5). R domains not only release the synthesised peptide and therefore regenerate the NRPS machinery, but also introduce functional/reactive groups (warheads) into the synthesised peptide by catalysing an NAD (P) H dependent two-electron reduction of the thioester to an aldehyde which can be further reduced to an alcohol59. Peptide aldehydes are often associated with protease inhibitors, for example, by reversible binding of the active site's threonine of the Mycobacterium tuberculosis proteasome by fellutamide B60.

However, inspired by the potent proteasome inhibitor fellutamide B, we joined building blocks of up to five different NRPSs from four different Photorhabdus and Xenorhabdus strains (FIG. 5). The resulting chimeric assembly line enzymes NRPS-4 to 8 were produced in E. coli DH10B::mtaA, and culture extracts analysed via HPLC-MS/MS. All created NRPSs showed catalytic activity producing the desired lipopeptide aldehydes 13-19.

When tested against the yeast proteasome, compounds 15 & 17 indeed showed the expected inhibitory activity (low UM range).

### Example 3: Turning NRPS into Artificial NRPS/PKS Hybrid Synthetases

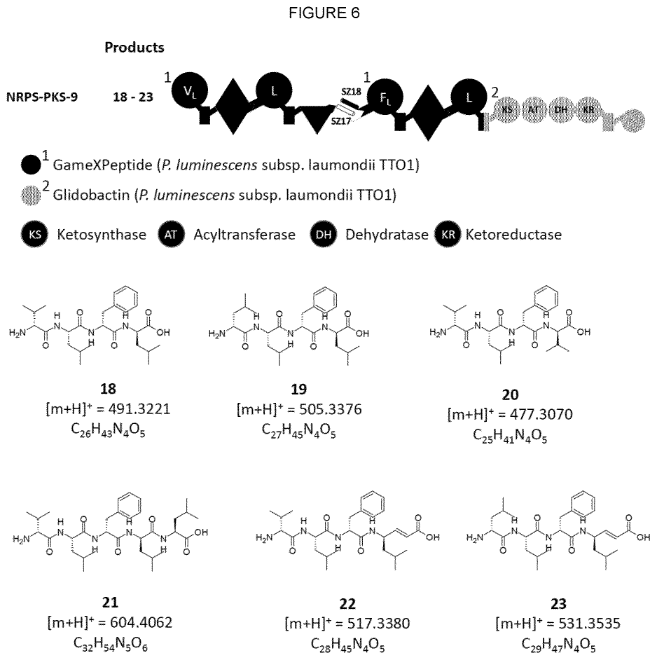

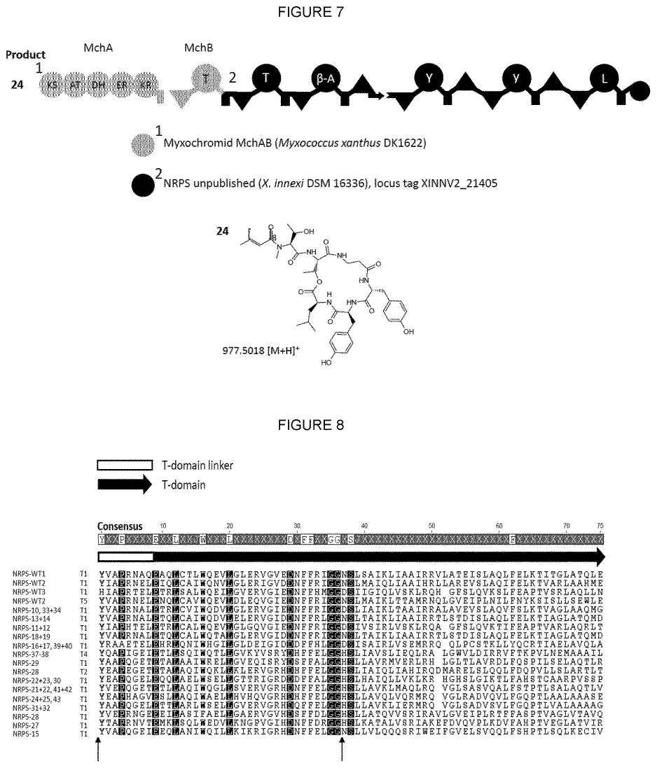

NRPS-PKS-9 (FIG. 6) and PKS-NRPS-10 (FIG. 7) were created to find out whether the identified fusion sites 3 & 1, respectively, could also be used to artificially fuse NRPS and PKS building blocks to create catalytically active NRPS/PKS hybrid synthetases.

Thus, once again, we have used our typeS GxpS3 model system to turn the NRPS into an artificial NRPS-PKS hybrid synthetase (FIG. 6). This was achieved by removing the GxpS termination unit (pT4-C/E5-A5-TE) and replacing it with a PKS termination unit (pT3-KS-AT-DHKR-ACP-TE) from the Glidobactin producing hybrid synthetase61. In brief, the resulting chimeric NRPS-PKS-9 synthesised 6 different peptides (20-25). Whereas peptides 20-23 do not carrying the desired terminal ketide group and are synthesised by the sole activity of the NRPS part, peptides 24 and 25 are the desired full-length hybrid NRPS-PKS products.

PKS-NRPS-10 is a further example highlighting how NRPS can be turned into NRPS-PKS hybrids (FIG. 7). In contrast to NRPS-PKS-9, here fusion point 1 was used to exchange the CstartA1 initiation unit of an unpublished cyclic lipopeptide producing NRPS (locus tag XINNV2_12405) from X. innexii DSM16336 against KS1-AT1-DH1-ER1-KR1-T1-C2-A2 of the Myxochromid62 producing synthetase (MchAB) from Myxococcus xanthus DK162263. Although both initiation units are derived from unrelated organisms with very different GC-contents (Xenorhabdus sp.: ~44%; Myxococcus sp.: ~70%), the resulting assembly line synthesised the desired PK-NRP hybrid product 26.

### Example 4: Recombining Non-Related Building Blocks of Varying GC-Content and Codon Usage to Create Chimeric NRPS

NRPS-PKS-9 and PKS-NRPS-10 have not only demonstrated that, for the first time, naturally occurring NRPS proteins can be functionally converted into NRPS-PKS hybrids, but PKS-NRPS-10 has also shown that unrelated building blocks with very different GC contents can be combined with each other.

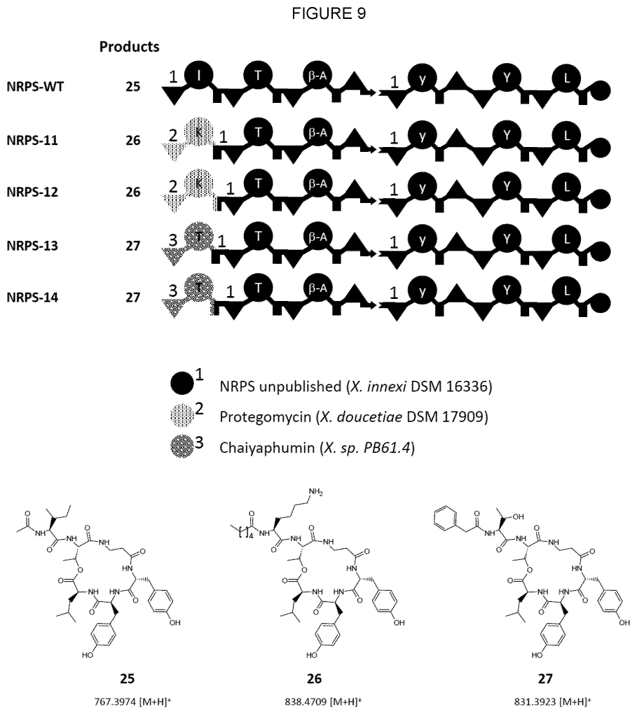

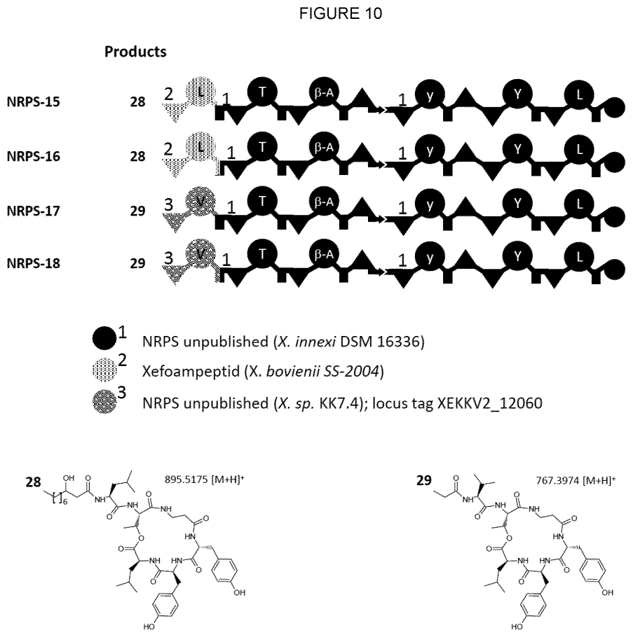

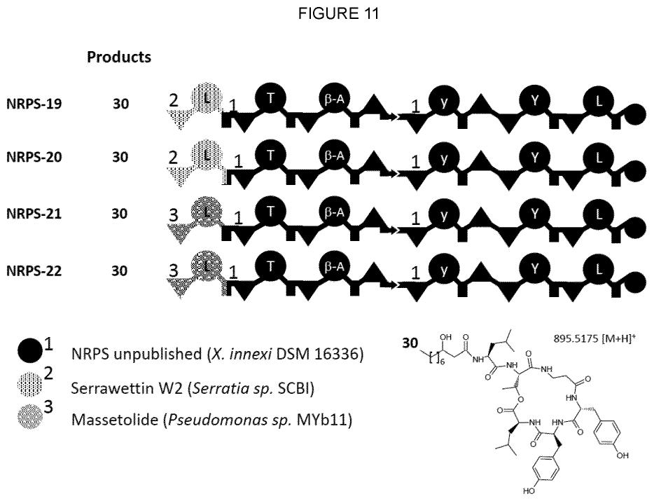

To further investigate and characterise the novel fusion points defined by the EIS-method we have created the chimeric proteins NRPS11-24 (FIG. 9-11). The aim was to combine the unpublished WT-NRPS-1 (X. innexii DSM16336, locus tag XINNV2_12405; FIG. 9) with initiation building blocks of Massetolide A64, Serrawettin W265, Serrawettin W166, Xefoampeptide67, unpublished NRPS (locus tag XEKKV2_12060), Protegomycin56, Chaiyaphumin68, and Plipastatin69 producing synthetases from Pseudomonas sp. MYb1170, Serratia sp. SCBI, S. marcescens DSM 12481, X. bovienii SS-2004, X. sp. KK7.4, X. doucetiae DSM 17909, X. sp. PB61.4, and B. subtilis 168, respectively. Additionally, each reprogrammed NRPS was designed and produced in two variants, applying both fusion sites 1 and 4. All resulting reprogrammed NRPS showed biocatalytic activity in both variants (fusion point 1 and 4), producing the desired cyclic lipopeptides (27-33).

### Conclusion:

It was possible to confirm the in vivo applicability of all evolutionary inspired and in silico detected fusion points (1, 3, & 4; FIG. 3) to modify and de novo generate synthetic NRPSs, and NRPS-PKS hybrids. Interestingly, all reprogrammed NRPSs and NRPS-PKS hybrids showed good to very good production titres comparable to and even exceeding WT production levels.

In general, all splicing positions 1, 3, & 4 can be used to functionally generate novel biosynthetic pathways. The great strength of the EIS method described above seems to be its generality, suggesting that recombination within T domains are not only a valid method for reprogramming NRPS in the laboratory for synthetic biology purposes—but that nature might actually use T domains as recombination points to insert and delete building blocks from existing BGCs or to recombine two BGCs via intragenomic homologous recombination. However, compared to all state-of-the-art methods 1-3,71, for the first time ever, it was possible to show that building blocks independent of their origin, preferred codon usage and GC-content of its originating strain functionally can be produced in E. coli to synthesise novel peptides and peptide-polyketide hybrids. Last but not least, compared to the total number of characterised extender units of all NRPSs and PKSs9, each bacterial species encodes for BGCs with only a very limited range of extender units. The EIS method thus allows us to expand the chemically available space beyond the limits set by nature.

### Example 5: Module Coding Library Generation

The strategy for library construction is illustrated in FIG. 3. T-domains in Organisms as diverse as Xenorhabdus, Pseudomonas and Burkholderia are not highly conserved across species. But when T-domains followed by a C- or CE-domain coded by one organism are compiled and aligned (FIG. 3A.) a conserved sequence spanning 10-12 amino acid can be observed. Compiled T-domains of the genera Xenorhabdus followed by E-domains show a different conserved sequence patch of even higher degree of sequence identity. Analysis of the underlying DNA sequence for this conserved amino acid patch allows the design of degenerated primers (FIG. 3B.) which potentially can target and amplify 70-90% of T-domains in organisms comprising more than 50 T-domains (e.g. Pseudomonas, Xenorhabdus, Burkholderia, Mycetohabitans) for which we evaluated this method.

Using the degenerated primer sets in PCR reactions with genomic DNA (gDNA) as template a library of module coding DNA fragments can be obtained. This library then is ligated into an entry vector. The ligation is performed with a dephosphorylated vector and 5′-phosphorylated PCR products. The entry vector codes at its ligation sites homology arms necessary for the Gibson cloning step (cloning based on stretches of homologous DNA regions) into the NRPS in the following steps (see FIGS. 3E. and 3F.). The initial PCR module library can be ligated into any entry vector flanked by any homology arm of choice. In order to clone the module library into the NRPS context a second round of PCR has to take place. The primer set for this PCR bind to the homology arms and overlap by 3 to 5 base pairs with the insert. The overlap of the primer pair with the ligated insert assures the sole amplification of ‘correctly’ ligated inserts (see FIG. 3E.). The Gibson cloning step (FIG. 3F.) inserts the module library into the NRPS biosynthesis of choice. The resulting clones can be used in production cultures in E. coli or any other appropriate prokaryotes. The resulting non-ribosomal peptides incorporate at the site of insertion of the module library diverse amino acids (FIG. 3G.). In the example given it is the third amino acid. It is important to note that the inserted module in position three defines the stereochemistry of the amino acid in position two and in doing so expands the number of possible peptides being produced.

### REFERENCES

The references are:
1. Bozhuyuk, K. A. J. et al. De novo design and engineering of non-ribosomal peptide synthetases. Nat Chem 10, 275-281, doi: 10.1038/nchem.2890 (2018).
2 Bozhuyuk, K. A. J. et al. Modification and de novo design of non-ribosomal peptide synthetases using specific assembly points within condensation domains. Nat Chem 11, 653-661, doi: 10.1038/s41557-019-0276-z (2019).
3 Bozhuyuk, K., Watzel, J., Abbood, N. & Bode, H. B. Synthetic Zippers as an Enabling Tool for Engineering of Non-Ribosomal Peptide Synthetases. Angew Chem Int Ed Engl, doi: 10.1002/anie.202102859 (2021).
4 Fierro, F. et al. Transcriptional and bioinformatic analysis of the 56.8 kb DNA region amplified in tandem repeats containing the penicillin gene cluster in Penicillium chrysogenum. Fungal Genet Biol 43, 618-629, doi: 10.1016/j.fgb.2006.03.001 (2006).
5 Li, J., Kim, S. G. & Blenis, J. Rapamycin: one drug, many effects. Cell Metab 19, 373-379, doi: 10.1016/j.cmet.2014.01.001 (2014).
6 Murray, V., Chen, J. K. & Chung, L. H. The Interaction of the Metallo-Glycopeptide Anti-Tumour Drug Bleomycin with DNA. Int J Mol Sci 19, doi: 10.3390/ijms 19051372 (2018).
7 Sussmuth, R. D. & Mainz, A. Nonribosomal Peptide Synthesis-Principles and Prospects. Angew Chem Int Ed Engl 56, 3770-3821, doi: 10.1002/anie.201609079 (2017).
8 Nivina, A., Yuet, K. P., Hsu, J. & Khosla, C. Evolution and Diversity of AssemblyLine Polyketide Synthases. Chem Rev 119, 12524-12547, doi: 10.1021/acs.chemrev.9b00525 (2019).
9 Flissi, A. et al. Norine: update of the nonribosomal peptide resource. Nucleic Acids Res 48, D465-D469, doi: 10.1093/nar/gkz1000 (2020).
10 Caboche, S., Leclere, V., Pupin, M., Kucherov, G. & Jacques, P. Diversity of monomers in nonribosomal peptides: towards the prediction of origin and biological activity. J Bacteriol 192, 5143-5150, doi: 10.1128/JB.00315-10 (2010).
11 Walsh, C. T., O'Brien, R. V. & Khosla, C. Nonproteinogenic amino acid building blocks for nonribosomal peptide and hybrid polyketide scaffolds. Angew Chem Int Ed Engl 52, 7098-7124, doi: 10.1002/anie.201208344 (2013).
12 Ray, L. & Moore, B. S. Recent advances in the biosynthesis of unusual polyketide synthase substrates. Nat Prod Rep 33, 150-161, doi: 10.1039/c5np00112a (2016).
13 Blin, K. et al. antiSMASH 4.0-improvements in chemistry prediction and gene cluster boundary identification. Nucleic Acids Res 45, W36-W41, doi: 10.1093/nar/gkx319 (2017).
14 Skinnider, M. A., Merwin, N. J., Johnston, C. W. & Magarvey, N. A. PRISM 3: expanded prediction of natural product chemical structures from microbial genomes. Nucleic Acids Res 45, W49-W54, doi: 10.1093/nar/gkx320 (2017).
15 Bozhuyuk, K. A., Micklefield, J. & Wilkinson, B. Engineering enzymatic assembly lines to produce new antibiotics. Curr Opin Microbiol 51, 88-96, doi: 10.1016/j.mib.2019.10.007 (2019).
16 Dutta, S. et al. Structure of a modular polyketide synthase. Nature 510, 512-517, doi: 10.1038/nature13423 (2014).
17 Whicher, J. R. et al. Structural rearrangements of a polyketide synthase module during its catalytic cycle. Nature 510, 560-564, doi: 10.1038/nature 13409 (2014).
18 Drake, E. J. et al. Structures of two distinct conformations of holo-non-ribosomal peptide synthetases. Nature 529, 235-238, doi: 10.1038/nature 16163 (2016).
19 Miller, B. R., Drake, E. J., Shi, C., Aldrich, C. C. & Gulick, A. M. Structures of a Nonribosomal Peptide Synthetase Module Bound to MbtH-like Proteins Support a Highly Dynamic Domain Architecture. J Biol Chem 291, 22559-22571, doi: 10.1074/jbc.M116.746297 (2016).
20 Jenke-Kodama, H. & Dittmann, E. Evolution of metabolic diversity: insights from microbial polyketide synthases. Phytochemistry 70, 1858-1866, doi: 10.1016/j.phytochem.2009.05.021 (2009).
21 Zhang, L. et al. Characterization of Giant Modular PKSs Provides Insight into Genetic Mechanism for Structural Diversification of Aminopolyol Polyketides. Angew Chem Int Ed Engl 56, 1740-1745, doi: 10.1002/anie.201611371 (2017).
22 Wlodek, A. et al. Diversity oriented biosynthesis via accelerated evolution of modular gene clusters. Nat Commun 8, 1206, doi: 10.1038/s41467-017-01344-3 (2017).
23 Baunach, M., Chowdhury, S., Stallforth, P. & Dittmann, E. The Landscape of Recombination Events That Create Nonribosomal Peptide Diversity. Mol Biol Evol 38, 2116-2130, doi: 10.1093/molbev/msab015 (2021).
24 Minin, V. N., Dorman, K. S., Fang, F. & Suchard, M. A. Dual multiple change-point model leads to more accurate recombination detection. Bioinformatics 21, 3034-3042, doi: 10.1093/bioinformatics/bti459 (2005).
25 Martin, D. P., Murrell, B., Golden, M., Khoosal, A. & Muhire, B. RDP4: Detection and analysis of recombination patterns in virus genomes. Virus Evol 1, vev003, doi: 10.1093/ve/vev003 (2015).
26 Sieber, S. A. & Marahiel, M. A. Molecular mechanisms underlying nonribosomal peptide synthesis: approaches to new antibiotics. Chem Rev 105, 715-738, doi: 10.1021/cr0301191 (2005).
27 Beer, R. et al. Creating functional engineered variants of the single-module nonribosomal peptide synthetase IndC by T domain exchange. Mol Biosyst 10, 1709-1718, doi: 10.1039/c3mb70594c (2014).
28 Koglin, A. & Walsh, C. T. Structural insights into nonribosomal peptide enzymatic assembly lines. Nat Prod Rep 26, 987-1000, doi: 10.1039/b904543k (2009).
29 Zhou, Z., Lai, J. R. & Walsh, C. T. Directed evolution of aryl carrier proteins in the enterobactin synthetase. Proc Natl Acad Sci USA 104, 11621-11626, doi: 10.1073/pnas.0705122104 (2007).
30 Finking, R. & Marahiel, M. A. Biosynthesis of nonribosomal peptides 1. Annu Rev Microbiol 58, 453-488, doi: 10.1146/annurev.micro.58.030603.123615 (2004).
31 Lai, J. R., Koglin, A. & Walsh, C. T. Carrier protein structure and recognition in polyketide and nonribosomal peptide biosynthesis. Biochemistry 45, 14869-14879, doi: 10.1021/bi061979p (2006).
32 Mofid, M. R., Finking, R., Essen, L. O. & Marahiel, M. A. Structure-based mutational analysis of the 4′-phosphopantetheinyl transferases Sfp from Bacillus subtilis: carrier protein recognition and reaction mechanism. Biochemistry 43, 4128-4136, doi: 10.1021/bi036013h (2004).
33 Mofid, M. R., Finking, R. & Marahiel, M. A. Recognition of hybrid peptidyl carrier proteins/acyl carrier proteins in nonribosomal peptide synthetase modules by the 4′-phosphopantetheinyl transferases AcpS and Sfp. J Biol Chem 277, 17023-17031, doi: 10.1074/jbc.M200120200 (2002).
34 Lambalot, R. H. et al. A new enzyme superfamily—the phosphopantetheinyl transferases. Chem Biol 3, 923-936, doi: 10.1016/s1074-5521 (96) 90181-7 (1996).
35 Walsh, C. T., Gehring, A. M., Weinreb, P. H., Quadri, L. E. & Flugel, R. S. Posttranslational modification of polyketide and nonribosomal peptide synthases. Curr Opin Chem Biol 1, 309-315, doi: 10.1016/s1367-5931 (97) 80067-1 (1997).
36 Quadri, L. E. et al. Characterization of Sfp, a Bacillus subtilis phosphopantetheinyl transferase for peptidyl carrier protein domains in peptide synthetases. Biochemistry 37, 1585-1595, doi: 10.1021/bi9719861 (1998).
37 Reuter, K., Mofid, M. R., Marahiel, M. A. & Ficner, R. Crystal structure of the surfactin synthetase-activating enzyme sfp: a prototype of the 4′-phosphopantetheinyl transferase superfamily. EMBO J 18, 6823-6831, doi: 10.1093/emboj/18.23.6823 (1999).
38 Weber, T., Baumgartner, R., Renner, C., Marahiel, M. A. & Holak, T. A. Solution structure of PCP, a prototype for the peptidyl carrier domains of modular peptide synthetases. Structure 8, 407-418, doi: 10.1016/s0969-2126 (00) 00120-9 (2000).
39 Holak, T. A., Kearsley, S. K., Kim, Y. & Prestegard, J. H. Three-dimensional structure of acyl carrier protein determined by NMR pseudoenergy and distance geometry calculations. Biochemistry 27, 6135-6142, doi: 10.1021/bi00416a046 (1988).
40 Parris, K. D. et al. Crystal structures of substrate binding to Bacillus subtilis holo(acyl carrier protein) synthase reveal a novel trimeric arrangement of molecules resulting in three active sites. Structure 8, 883-895, doi: 10.1016/s0969-2126 (00) 00178-7 (2000).
41 Crump, M. P. et al. Solution structure of the actinorhodin polyketide synthase acyl carrier protein from Streptomyces coelicolor A3 (2). Biochemistry 36, 6000-6008, doi: 10.1021/bi970006+ (1997).
42 Koglin, A. et al. Conformational switches modulate protein interactions in peptide antibiotic synthetases. Science 312, 273-276, doi: 10.1126/science. 1122928 (2006).
43 Kim, Y., Ohlrogge, J. B. & Prestegard, J. H. Motional effects on NMR structural data. Comparison of spinach and Escherichia coli acyl carrier proteins. Biochem Pharmacol 40, 7-13, doi: 10.1016/0006-2952 (90) 90171-g (1990).
44 Zornetzer, G. A., Fox, B. G. & Markley, J. L. Solution structures of spinach acyl carrier protein with decanoate and stearate. Biochemistry 45, 5217-5227, doi: 10.1021/bi052062d (2006).
45 Alekseyev, V. Y., Liu, C. W., Cane, D. E., Puglisi, J. D. & Khosla, C. Solution structure and proposed domain domain recognition interface of an acyl carrier protein domain from a modular polyketide synthase. Protein Sci 16, 2093-2107, doi: 10.1110/ps.073011407 (2007).
46 Weber, T. et al. antiSMASH 3.0-a comprehensive resource for the genome mining of biosynthetic gene clusters. Nucleic Acids Res 43, W237-243, doi: 10.1093/nar/gkv437 (2015).
47 Findlow, S. C., Winsor, C., Simpson, T. J., Crosby, J. & Crump, M. P. Solution structure and dynamics of oxytetracycline polyketide synthase acyl carrier protein from Streptomyces rimosus. Biochemistry 42, 8423-8433, doi: 10.1021/bi0342259 (2003).
48 Johnson, M. A., Peti, W., Herrmann, T., Wilson, I. A. & Wuthrich, K. Solution structure of Asl1650, an acyl carrier protein from Anabaena sp. PCC 7120 with a variant phosphopantetheinylation-site sequence. Protein Sci 15, 1030-1041, doi: 10.1110/ps.051964606 (2006).
49 Sharma, A. K., Sharma, S. K., Surolia, A., Surolia, N. & Sarma, S. P. Solution structures of conformationally equilibrium forms of holo-acyl carrier protein (PfACP) from Plasmodium falciparum provides insight into the mechanism of activation of ACPs. Biochemistry 45, 6904-6916, doi: 10.1021/bi060368u (2006).
50 Kim, Y. & Prestegard, J. H. A dynamic model for the structure of acyl carrier protein in solution. Biochemistry 28, 8792-8797, doi: 10.1021/bi00448a017 (1989).
51 Tanovic, A., Samel, S. A., Essen, L. O. & Marahiel, M. A. Crystal structure of the termination module of a nonribosomal peptide synthetase. Science 321, 659-663, doi: 10.1126/science. 1159850 (2008).
52 Grigoriev, A. Analyzing genomes with cumulative skew diagrams. Nucleic Acids Res 26, 2286-2290, doi: 10.1093/nar/26.10.2286 (1998).
53 Lobry, J. R. Asymmetric substitution patterns in the two DNA strands of bacteria. Mol Biol Evol 13, 660-665, doi: 10.1093/oxfordjournals.molbev.a025626 (1996).
54 Nollmann, F. I. et al. Insect-specific production of new GameXPeptides in photorhabdus luminescens TTO1, widespread natural products in entomopathogenic bacteria. Chembiochem 16, 205-208, doi: 10.1002/cbic.201402603 (2015).
55 Pantel, L. et al. Odilorhabdins, Antibacterial Agents that Cause Miscoding by Binding at a New Ribosomal Site. Mol Cell 70, 83-94 e87, doi: 10.1016/j.molcel.2018.03.001 (2018).
56 Behsaz, B. et al. Integrating genomics and metabolomics for scalable non-ribosomal peptide discovery. Nat Commun 12, 3225, doi: 10.1038/s41467-021-23502-4 (2021).
57 Du, L. & Lou, L. PKS and NRPS release mechanisms. Nat Prod Rep 27, 255-278, doi: 10.1039/b912037h (2010).
58 Tietze, A., Shi, Y. N., Kronenwerth, M. & Bode, H. B. Nonribosomal Peptides Produced by Minimal and Engineered Synthetases with Terminal Reductase Domains. Chembiochem 21, 2750-2754, doi: 10.1002/cbic.202000176 (2020).
59 Chhabra, A. et al. Nonprocessive [2+2] e-off-loading reductase domains from mycobacterial nonribosomal peptide synthetases. Proc Natl Acad Sci USA 109, 5681-5686, doi: 10.1073/pnas. 1118680109 (2012).
60 Lin, G., Li, D., Chidawanyika, T., Nathan, C. & Li, H. Fellutamide B is a potent inhibitor of the Mycobacterium tuberculosis proteasome. Arch Biochem Biophys 501, 214-220, doi: 10.1016/j.abb.2010.06.009 (2010).
61 Oka, M. et al. Chemical modification of the antitumor antibiotic glidobactin. J Antibiot (Tokyo) 41, 1812-1822, doi: 10.7164/antibiotics.41.1812 (1988).
62 Wenzel, S. C. et al. Structure and biosynthesis of myxochromides S1-3 in Stigmatella aurantiaca: evidence for an iterative bacterial type I polyketide synthase and for module skipping in nonribosomal peptide biosynthesis. Chembiochem 6, 375-385, doi: 10.1002/cbic.200400282 (2005).
63 Krug, D. et al. Discovering the hidden secondary metabolome of Myxococcus xanthus: a study of intraspecific diversity. Appl Environ Microbiol 74, 3058-3068, doi: 10.1128/AEM.02863-07 (2008).
64 de Bruijn, I., de Kock, M. J., de Waard, P., van Beek, T. A. & Raaijmakers, J. M. Massetolide A biosynthesis in Pseudomonas fluorescens. J Bacteriol 190, 2777-2789, doi: 10.1128/JB.01563-07 (2008).
65 Petersen, L. M., LaCourse, K., Schoner, T. A., Bode, H. & Tisa, L. S. Inactivation of the Major Hemolysin Gene Influences Expression of the Nonribosomal Peptide Synthetase Gene swrA in the Insect Pathogen Serratia sp. Strain SCBI. J Bacteriol 199, doi: 10.1128/JB.00333-17 (2017).
66 Li, H., Tanikawa, T., Sato, Y., Nakagawa, Y. & Matsuyama, T. Serratia marcescens gene required for surfactant serrawettin W1 production encodes putative aminolipid synthetase belonging to nonribosomal peptide synthetase family. Microbiol Immunol 49, 303-310, doi: 10.1111/j. 1348-0421.2005.tb03734.x (2005).
67 Kegler, C. & Bode, H. B. Artificial Splitting of a Non-Ribosomal Peptide Synthetase by Inserting Natural Docking Domains. Angew Chem Int Ed Engl 59, 13463-13467, doi: 10.1002/anie.201915989 (2020).
68 Grundmann, F. et al. Antiparasitic chaiyaphumines from entomopathogenic Xenorhabdus sp. PB61.4. J Nat Prod 77, 779-783, doi: 10.1021/np4007525 (2014).
69 Batool, M., Khalid, M. H., Hassan, M. N. & Fauzia Yusuf, H. Homology modeling of an antifungal metabolite plipastatin synthase from the Bacillus subtilis 168. Bioinformation 7, 384-387, doi: 10.6026/97320630007384 (2011).
70 Kissoyan, K. A. B. et al. Natural C. elegans Microbiota Protects against Infection via Production of a Cyclic Lipopeptide of the Viscosin Group. Curr Biol 29, 1030-1037 e1035, doi: 10.1016/j.cub.2019.01.050 (2019).
71 Calcott, M. J., Owen, J. G. & Ackerley, D. F. Efficient rational modification of non-ribosomal peptides by adenylation domain substitution. Nat Commun 11, 4554, doi: 10.1038/s41467-020-18365-0 (2020).
72 Zhou, Q. et al. Structure and biosynthesis of xenoamicins from entomopathogenic Xenorhabdus. Chemistry 19, 16772-16779, doi: 10.1002/chem.201302481 (2013).

1. A chimeric protein for the production of non-ribosomal peptides, comprising at least one chimeric thiolation domain (T-domain (PKS acyl carrier protein (ACP) or NRPS petidyl carrier (PCP))) consisting of a first T-domain (PKS acyl carrier protein (ACP) or NRPS petidyl carrier (PCP)) segment directly adjacent to a second T-domain (PKS acyl carrier protein (ACP) or NRPS petidyl carrier (PCP)) segment, wherein the first T-domain (PKS acyl carrier protein (ACP) or NRPS petidyl carrier (PCP)) segment comprises an amino acid sequence from an N-terminal part of a T-domain (PKS acyl carrier protein (ACP) or NRPS petidyl carrier (PCP)) amino acid sequence, and wherein the second T-domain (PKS acyl carrier protein (ACP) or NRPS petidyl carrier (PCP)) segment comprises an amino acid sequence from a C-terminal part of a T-domain (PKS acyl carrier protein (ACP) or NRPS petidyl carrier (PCP)) amino sequence; wherein the chimeric thiolation domain is a fully functional T-domain (PKS acyl carrier protein (ACP) or NRPS petidyl carrier (PCP)); characterized in that
the amino acid sequences of the parts of the T-domain (PKS acyl carrier protein (ACP) or NRPS petidyl carrier (PCP)) amino sequences of the first and the second T-domain (PKS acyl carrier protein (ACP) or NRPS petidyl carrier (PCP)) segments are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.

2. The chimeric protein of claim 1, wherein the first T-domain (PKS acyl carrier protein (ACP) or NRPS petidyl carrier (PCP)) segment is directly adjacent to, and connected with, the N-terminal end of the second T-domain (PKS acyl carrier protein (ACP) or NRPS petidyl carrier (PCP)) segment.

3. The chimeric protein of claim 1, wherein the chimeric protein is a chimeric non-ribosomal peptide synthetases (NRPSs), a chimeric polyketide synthase (PKSs), or a chimeric NRPS/PKS hybrid.

4. The chimeric protein of claim 1, further comprising one or more known NRPS and/or PKS domains, in particular any or any combination of Adenylation (A) domain(s), Condensation (C) domain(s), Epimerization (E) domain(s), Reductase (R) domain(s), C/E domain(s), Oxidation (Ox) domain(s), Reduktion (Red) domain(s), hetero cyclisation (Cy) domain(s), starter C domain(s), Methylation (Mt) domain(s), A-Mt domain(s), A-Ox domain(s), Communication (Com) domain(s), Formylation (F) domain(s), X (X) domains, and/or terminal thioesterase (TE) domain(s) or terminal C domains; and/or the PKS domains Acyltransferase (AT) domain(s), Ketosynthase (KS) domain(s), Dehydratase (DH) domain(s), Ketoreductase (KR) domain(s), Enoylreductase (ER) domain(s), Methyltransferase (Mt) O- or C-domain(s), Docking (DD) domain(s), Thioesterase (TE) domain(s), PLP-dependent cysteine lyase (SH) domain(s), Acylcarrierprotein (ACP).

5. The chimeric protein of claim 1, wherein the chimeric T domain consists of the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence.

6. The chimeric protein of claim 1, wherein the N-terminal T-domain (PKS acyl carrier protein (ACP) or NRPS petidyl carrier (PCP)) amino acid sequence and the C-terminal T-domain (PKS acyl carrier protein (ACP) or NRPS petidyl carrier (PCP)) amino sequence are fused together at a fusion site, and wherein said fusion site is located +/−5 amino acids around the fusion sites 1 to 7 indicated in FIG. 3, preferably fusion sites 3 and 4.

7. The chimeric protein of claim 6, wherein the fusion site is directly before or after any amino acid position of the conserved T domain motif-FFxxGG (H/N/D)S-, wherein x is any amino acid.

8. The chimeric protein of claim 1, wherein in claim 1 (a) the protein further comprises at least one NRPS/PKS domain N-terminal of the chimeric T domain derived from a first microorganism species, and at least one NRPS/PKS domain C-terminal of the chimeric T domain and which is derived from a second microorganism species, with the provision that the first and the second microorganism species are not identical.

9. The chimeric protein of claim 1, wherein the chimeric protein comprises a sequence of NRPS/PKS domains that is capable of catalysing peptide and/or hybrid peptide-polyketide synthesis.

10. A system for the generation of chimeric non-ribosomal peptide synthetases (NRPSs), a chimeric polyketide synthase (PKSs), or a chimeric NRPS/PKS hybrid, comprising at least two system units:
(a) a first system unit comprising at least the first T-domain (PKS acyl carrier protein (ACP) or NRPS petidyl carrier (PCP)) segment recited in claim 1, and
(b) a second system unit comprising at least the second T domain segment;
wherein each system unit may comprise one or more of further NRPS/PKS domains, and wherein the chimeric non-ribosomal peptide synthetases (NRPSs), the chimeric polyketide synthase (PKSs), or the chimeric NRPS/PKS hybrid is obtainable by fusing the first and the second system unit such that the N-terminal T-domain (PKS acyl carrier protein (ACP) or NRPS petidyl carrier (PCP)) amino acid sequence of the first system unit is directly fused to the C-terminal T-domain (PKS acyl carrier protein (ACP) or NRPS petidyl carrier (PCP)) amino acid sequence of the second system unit.

11. The system of claim 10, wherein the sequences of the first system unit and the second system unit comprise amino acid sequences of the parts of the T-domain (PKS acyl carrier protein (ACP) or NRPS petidyl carrier (PCP)) amino sequences of the first and the second T-domain (PKS acyl carrier protein (ACP) or NRPS petidyl carrier (PCP)) segments, which are:
(a) T-domain (PKS acyl carrier protein (ACP) or NRPS petidyl carrier (PCP)) sequences that are derived from different species which are heterologous to each other; or
(b) T-domain (PKS acyl carrier protein (ACP) or NRPS petidyl carrier (PCP)) sequence that are derived from different NRPS/PKS genes of the same species; or
(c) T-domain (PKS acyl carrier protein (ACP) or NRPS petidyl carrier (PCP)) sequences that are derived from two different T-domains (PKS acyl carrier protein (ACP) or NRPS petidyl carrier (PCP)) of the same NRPS/PKS gene.

12. A method for the generation of chimeric non-ribosomal peptide synthetases (NRPSs), a chimeric polyketide synthase (PKSs), or a chimeric NRPS/PKS hybrid, the method comprising the steps of:
(a) Providing a first NRPS or PKS gene sequence from a first microorganism species,
(b) Providing a second NRPS or PKS gene sequence from a second microorganism species,
(c) Combining at least one part of the first NRPS or PKS gene sequence with at least one part of the second NRPS or PKS gene sequence to obtain a chimeric gene sequence by fusing both sequences to each other such that at least one fusion point is located within a T domain, and wherein the fusion product comprises a T domain consisting of an N-terminal sequence of a T domain of the first NRPS or PKS gene sequence and a C-terminal sequence of a T domain of the second NRPS or PKS gene sequence, and
(d) Optionally, expressing the chimeric gene sequence to obtain the chimeric nonribosomal peptide synthetases (NRPSs), a chimeric polyketide synthase (PKSs), or a chimeric NRPS/PKS hybrid.

13. A method for combining, or producing a combination of, at least two NRPS/PKS modules from two different NRPS/PKS genes or from two separated locations within the same NRPS/PKS gene, the method comprising the steps of:
(a) Identifying a first target NRPS/PKS module that is to be combined directly N-terminally to a second target NRPS/PKS module;
(b) Identifying a second target NRPS/PKS module that is to be combined directly C-terminally to a second target NRPS/PKS module, and wherein the second target NRPS/PKS module is selected from a module that is located within a NRPS/PKS gene C-terminally to a third non-target NRPS/PKS module;
(c) Combining the first target NRPS/PKS module with the second target NRPS/PKS module by directly attaching an N-terminal part of the T domain of the first NRPS/PKS module with a C-terminal part of a T domain of the third non-target NRPS/PKS module;
Wherein the combination of the N-terminal part of the T domain of the first NRPS/PKS module with the C-terminal part of a T domain of the third non-target NRPS/PKS module constitute a full and functional T-domain (PKS acyl carrier protein (ACP) or NRPS petidyl carrier (PCP)).

14. The method of claim 13, wherein the first and the third NRPS/PKS modules each comprise at least a thiolation domain.

15. (canceled)

16. (canceled)

17. The method of claim 13, wherein the first target NRPS/PKS module and the third non-target NRPS/PKS module are not identical.

18. A combination product obtained or obtainable by the method of claim 13.

19. A method for the generation of a nucleic acid construct encoding a system unit of claim 10, the method comprising a step of amplifying a sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S, wherein x is any amino acid.

20. A method for generating a library of nucleic acid constructs each encoding a system unit of claim 10, wherein the method comprises a step of amplifying a first sequence stretch of an NRPS/PKS gene using a degenerate primer pair which hybridizes under stringent conditions to at least 10 nucleic acids of a hybridization site within the NRPS/PKS gene, wherein the hybridization site comprises a sequence encoding for an amino acid consensus motif (T domain motif) according to SEQ ID NO: XY: FFxxGG(H/N/D)S; and
wherein the method comprises a second amplification of at least a second sequence stretch of an NRPS/PKS gene using the degenerate primer pair from the same or one or more different NRPS/PKS genes.

21. A method for producing a chimeric non-ribosomal peptide synthetase (NRPS)- and/or polyketide (PKS)-module, the method comprising:
(a) Providing a first NRPS/PKS module sequence comprising at least a first T-domain (T-domain (PKS acyl carrier protein (ACP) or NRPS petidyl carrier (PCP)) sequence;
(b) Providing a second NRPS/PKS module sequence comprising at least a second T-domain sequence;
(c) Optionally, aligning the first T-domain sequence to the second T-domain sequence to identify at least one T-domain fusion point;
(d) Fusing by cloning directly adjacent to each other into a genetic construct a first partial sequence to a second partial sequence to obtain a chimeric module sequence, wherein
i. The first partial sequence is a nucleotide sequence encoding an amino acid sequence of the first NRPS/PKS module sequence located directly N-terminal to the identified T-domain fusion point; and
ii. The second partial sequence is a nucleotide sequence encoding an amino acid sequence of the second NRPS/PKS module sequence located directly C-terminal to the identified T-domain fusion point;
and thereby producing a genetic construct comprising a chimeric NRPS- and/or PKS-module.

22. The method of claim 21, wherein the first NRPS/PKS module sequence and the second NRPS/PKS module sequence are not identical, preferably wherein the first T-domain sequence and the second T-domain sequence are not identical.

23. The method of claim 21, wherein the first NRPS/PKS module sequence and the second NRPS/PKS module sequence are derived from the same NRPS/PKS gene, but are not directly adjacent to each other.

24. The method of claim 21, wherein the first NRPS/PKS module sequence and the second NRPS/PKS module sequence are derived from two different NRPS/PKS genes; preferably of two different bacterial and/or fungal species.

25. (canceled)

26. The method of claim 21, wherein the T-domain fusion point is selected to be positioned at one of the following positions of the aligned sequences with reference to the T-domain structure comprising from N- to C-terminus: T-domain linker domain, Helix-1, Loop-1, Helix-2, Loop-2, Helix-3, Helix-4, wherein
(a) Within or N-terminal of the T-domain linker domain,
(b) Within the Loop-1 domain;
(c) Within the Helix-2;
(d)C-terminal of the Helix-4.

27. The method of claim 21, further comprising expressing the chimeric NRPS- and/or PKS-module from the genetic construct.

28. (canceled)


SEQUENCE LISTING (1 sequence)

SEQ ID NO: 1 - protein, 8 aa, synthetic construct
  UNSURE 3..4: note = Residues 3 and 4 can be any amino acid
  UNSURE 7: note = Residue 7 can be selected from H, N or D
FFXXGGXS